(19) United States
(12) Patent Application Publication
(10) Pub. No.: US 2026/0221277 A1
(43) Pub. Date: Jul. 30, 2026

(54) METHODS AND SYSTEMS FOR ACTIVITY DETECTION AND QUANTIFICATION OF MOVEMENT KINEMATICS

(71) Applicant: President and Fellows of Harvard College, Cambridge, MA (US)

(72) Inventors: Dmitry Popov, Cambridge, MA (US); Conor J. Walsh, Cambridge, MA (US); Daekyum Kim, Cambridge, MA (US); Haedo Cho, Cambridge, MA (US); Francesco Bertacchi, Cambridge, MA (US)

(73) Assignee: President and Fellows of Harvard College, Cambridge, MA (US)

(21) Appl. No.: 19/147,306

(22) PCT Filed: Jan. 10, 2024
(86) PCT No.: PCT/US2024/011111

§ 371 Date: Jul. 10, 2025

Related U.S. Application Data
(60) Provisional application No. 63/438,460, filed on Jan. 11, 2023.
(60) Provisional application No. 63/438,465, filed on Jan. 11, 2023.

(51) Int. Cl. G16H 40/63

(52) U.S. Cl. CPC G16H 40/63

(57) ABSTRACT

The techniques described herein relate to computerized methods and systems for determining a type and/or kinematic characteristics of activities performed by a subject using one or more statistical models and training of such models. In some embodiments, a system may receive sensor data from one or more sensors indicating movement of one or more body portions of a subject, and using an activity identification statistical model to determine a type of activity. In some embodiments, a system may receive sensor data from the one or more sensors, and use a kinematic characteristic statistical model to determine one or more kinematic characteristics of the activity based on the sensor data. In some embodiments, the output of the activity identification statistical model and/or the output from the kinematic characteristic statistical model may be used with the other model. In some embodiments, the above concepts may be implemented using a single statistical model.

### RELATED APPLICATIONS

This application claims the benefit of priority under 35 U.S.C. § 119 (e) of U.S. Provisional Application Ser. No. 63/438,460, filed Jan. 11, 2023, and U.S. Provisional Application Ser. No. 63/438,465, filed Jan. 11, 2023, the disclosures of which are incorporated herein by reference in their entirety.

### STATEMENT REGARDING FEDERALLY SPONSORED RESEARCH OR DEVELOPMENT

This invention was made with government support under 2129012 awarded by the National Science Foundation (NSF). The government has certain rights in this invention.

### FIELD

Disclosed embodiments are related to exercise detection and quantifying movement kinematics during exercise of a subject.

### BACKGROUND

Wearable technologies are used in conventional systems for monitoring and assessing movement of body parts of a subject. In these systems, a plurality of wearable sensors are worn on the subject. The sensor data from the plurality of wearable sensors are used to assess kinematic characteristics of the movement without consideration to which movements are associated with a specific exercise or not.

### SUMMARY

According to some embodiments, a system for activity identification and characterizing movement kinematics is provided. The system includes at least one processor configured to: receive sensor data from two or more sensors indicating movement of two or more body portions of a subject during movement of the subject; use a statistical model to determine a type of activity during the movement of the subject, based in part on the sensor data; and use the statistical model to determine one or more kinematic characteristics during the movement of the subject, based in part on the sensor data and the type of activity.

According to any of the above embodiments, the statistical model simultaneously determines the type of activity and the one or more kinematic characteristics. According to any of the above embodiments, the activity is an exercise.

According to any of the above embodiments, the two or more sensors are attachable to one or more pieces of exercise equipment.

According to any of the above embodiments, the two or more sensors are wearable on one or more body portions of the subject.

According to any of the above embodiments, the sensor data includes at least first data and second data respectively received from the at least two sensors, wherein the first data and the second data are time-synchronized.

According to any of the above embodiments, the at least two sensors include a first sensor attached to a proximal portion of the subject and a second sensor attached to a distal portion of the subject.

According to any of the above embodiments, the proximal portion of the subject includes a torso, a thigh, or a proximal portion of a limb of the subject; and the distal portion of the subject includes an end of a limb of the subject.

According to any of the above embodiments, the sensor data includes at least a difference between a first reading from the first sensor and a second reading from the second sensor.

According to any of the above embodiments, the at least one processor is further configured to: use the statistical model to additionally determine at least a start point and/or a stop point during the movement of the subject.

According to any of the above embodiments, wherein the sensor data includes at least one or more of: acceleration, angular velocity, or magnetometer data.

According to any of the above embodiments, the statistical model includes a dilated convolutional neural network.

According to any of the above embodiments, the statistical model, after determining the type of activity and the one or more kinematic characteristics, redetermines the type of activity based in part on the one or more kinematic characteristics.

According to any of the above embodiments, the statistical model determines the type of activity from a predetermined list of exercises.

According to any of the above embodiments, the statistical model determines a start point and/or a stop point of an activity.

According to any of the above embodiments, the at least one processor is configured to determine the one or more kinematic characteristics by at least estimating movement velocity and trajectory from the sensor data.

According to any of the above embodiments, the at least one processor is configured to estimate the movement velocity and trajectory from the sensor data by: estimating the movement velocity based on the sensor data; filtering the estimated movement velocity by removing signals with frequencies less than a threshold frequency; and estimating the movement trajectory based on the filtered movement velocity.

According to any of the above embodiments, the at least one processor is further configured to use the statistical model to determine the type of activity during the movement of the subject and/or to determine one or more kinematic characteristics during the movement of the subject, based in part on the movement velocity and trajectory.

According to any of the above embodiments, the at least one processor is configured to filter the estimated movement velocity by using a movement kinematic machine learning model.

According to any of the above embodiments, the one or more kinematic characteristics include one or more of: a number of repetitions, a pace of repetitions, an overall consistency, a velocity, a trajectory, and/or a range of motion.

According to any of the above embodiments, the statistical model includes an exercise identification statistical model and a kinematic characteristics statistical model.

According to any of the above embodiments, one or more layers of the exercise identification statistical model are connected to one or more layers of the kinematic characteristics statistical model.

According to any of the above embodiments, the exercise identification statistical model and the kinematic characteristics statistical model each include a respective dilated convolutional neural network.

According to some embodiments, a method for activity identification and characterizing movement kinematics is provided. The method includes by at least one processor: receiving sensor data from two or more sensors, the sensor data indicating movement of two or more body portions of a subject during movement of the subject; using a statistical model to determine a type of activity during the movement of the subject based in part on the sensor data; and using the statistical model to determine one or more kinematic characteristics during the movement of the subject, based in part on the sensor data and type of activity.

According to any of the above embodiments, the statistical model simultaneously determines the type of activity and the one or more kinematic characteristics.

According to any of the above embodiments, the two or more sensors are attachable to one or more pieces of exercise equipment.

According to any of the above embodiments, the two or more sensors are wearable on one or more body portions of the subject.

According to any of the above embodiments, the sensor data includes at least first data and second data respectively received from the at least two sensors, wherein the first data and the second data are time-synchronized.

According to any of the above embodiments, the at least two sensors include a first sensor attached to a proximal portion of the subject and a second sensor attached to a distal portion of the subject.

According to any of the above embodiments, the proximal portion of the subject includes a torso, a thigh, or a proximal portion of a limb of the subject; and the distal portion of the subject includes an end of a limb of the subject.

According to any of the above embodiments, the sensor data includes at least a difference between a first reading from the first sensor and a second reading from the second sensor.

According to any of the above embodiments, using the statistical model to additionally determine at least a start point and/or a stop point during the movement of the subject.

According to any of the above embodiments, the sensor data includes at least one or more of: acceleration, angular velocity, or magnetometer data.

According to any of the above embodiments, the method further includes, after using the statistical model to determine the type of activity and the one or more kinematic characteristics, using the statistical model to redetermine the type of activity based in part on the one or more kinematic characteristics.

According to any of the above embodiments, the type of activity is determined from a predetermined list of exercises.

According to any of the above embodiments, using the statistical model to additionally determine at least a start point and/or a stop point includes using the statistical model to determine a start point and/or a stop point of an activity.

According to any of the above embodiments, using the statistical model to determine one or more kinematic characteristics includes estimating movement velocity and trajectory from the sensor data.

According to any of the above embodiments, estimating movement velocity and trajectory includes: estimating the movement velocity based on the sensor data; filtering the estimated movement velocity by removing signals with frequencies less than a threshold frequency; and estimating the movement trajectory based on the filtered movement velocity.

According to any of the above embodiments, the method further includes using the statistical model to determine the type of activity during the movement of the subject and/or one or more kinematic characteristics during the movement of the subject, based in part on the movement velocity and trajectory.

According to any of the above embodiments, the method further includes filtering the estimated movement velocity using a kinematic characteristics statistical model.

According to any of the above embodiments, the one or more kinematic characteristics include one or more of: a number of repetitions, a pace of repetitions, an overall consistency, a velocity, a trajectory, and/or a range of motion.

According to any of the above embodiments, the statistical model includes an exercise identification statistical model and a kinematic characteristics statistical model.

According to any of the above embodiments, one or more layers of the exercise identification statistical model are connected to one or more layers of the kinematic characteristics statistical model.

According to any of the above embodiments, the exercise identification statistical model and the kinematic characteristics statistical model each include a respective dilated convolutional neural network.

According to some embodiments, a method for training a machine learning model configured to determine at least a type of activity from a movement of a subject based on sensor data associated with the movement of the subject is provided. The method includes receiving training sensor data collected at two or more sensors, the training sensor data indicating movement of two or more body portions of one or more subjects while performing one or more activities; obtaining data indicating at least a type of activity and one or more kinematic characteristics performed by the one or more subjects while the training sensor data is collected; and training the machine learning model to be configured to determine at least a type of activity and one or more kinematic characteristics from a movement of a subject, by using the received training sensor data and the data indicating the at least a type of activity and one or more kinematic characteristics associated with the training sensor data.

According to any of the above embodiments, training the machine learning model includes training an exercise identification statistical model to determine the type of activity from the movement of the subject by using the received training sensor data and the data indicating the type of activity associated with the training sensor data.

According to any of the above embodiments, the method further includes after training the exercise identification statistical model, locking weights of the exercise identification model; and training a kinematic characteristics statistical model to determine the one or more kinematic characteristics from the movement of the subject and the data indicating the one or more kinematic characteristics associated with the training sensor data.

According to any of the above embodiments, the method further includes after training the kinematic characteristics statistical model, unlocking the weights of the exercise identification model; and training the exercise identification statistical model and the kinematic characteristics statistical model using the received training sensor data and the data indicating the at least a type of activity and one or more kinematic characteristics associated with the training sensor data.

According to any of the above embodiments, the received training sensor data includes raw data recorded from an IMU sensor.

According to any of the above embodiments, the received training sensor data includes filtered data recorded from an IMU sensor.

According to any of the above embodiments, the two or more sensors are attachable to one or more pieces of exercise equipment.

According to any of the above embodiments, the two or more sensors are wearable on two or more body portions of the one or more subjects.

According to any of the above embodiments, the training sensor data includes at least first training data and second training data respectively received from the at least two sensors, wherein the first training data and the second training data are time-synchronized.

According to any of the above embodiments, the training sensor data includes at least a difference between corresponding values in the first training data and the second training data.

According to any of the above embodiments, obtaining training start/stop data including one or more start points and/or one or more stop points in the training sensor data, wherein the one or more start points each indicates a start of an activity when the training sensor data is collected, and wherein the one or more stop points each indicates a stop of an activity when the training sensor data is collected; and training the machine learning model to additionally determine at least a start point and/or a stop point during the movement of the subject, by additionally using the training start/stop data.

According to any of the above embodiments, obtaining the training start/stop data includes timing a start and/or stop of an activity performed by a respective subject of the one or more subjects when the training sensor data indicating movement of the respective subject is collected.

According to any of the above embodiments, the training sensor data includes at least one or more of: acceleration, angular velocity, or magnetometer data.

According to any of the above embodiments, the machine learning model includes a dilated convolutional neural network.

According to any of the above embodiments, the method further includes training the machine learning model to be configured to redetermine the type of activity based in part on the one or more kinematic characteristics.

According to any of the above embodiments, training the machine learning model includes training the machine learning model to be configured to determine the type of activity from a predetermined list of exercises.

According to any of the above embodiments, training the machine learning model to be configured to determine at least the type of activity and one or more kinematic characteristics includes training the machine learning model to estimate movement velocity and trajectory.

According to any of the above embodiments, training the machine learning model to estimate movement velocity and trajectory includes: training the machine learning model to estimate the movement velocity based on the training sensor data and data indicating one or more kinematic characteristics; filtering the estimated movement velocity by removing signals with frequencies less than a threshold frequency; and training the machine learning model to estimate the movement trajectory based on the filtered movement velocity and the data indicating one or more kinematic characteristics.

According to any of the above embodiments, the method further includes training the machine learning model to determine the type of activity during the movement of the subject and/or one or more kinematic characteristics during the movement of the subject, based in part on the movement velocity and trajectory.

According to any of the above embodiments, the one or more kinematic characteristics include one or more of: a number of repetitions, a pace of repetitions, an overall consistency, a velocity, a trajectory, and/or a range of motion.

According to any of the above embodiments, the machine learning model includes an exercise identification statistical model and a kinematic characteristics statistical model.

According to any of the above embodiments, one or more layers of the exercise identification statistical model are connected to one or more layers of the kinematic characteristics statistical model.

According to any of the above embodiments, the exercise identification statistical model and the kinematic characteristics statistical model each include a respective dilated convolutional neural network.

According some embodiments, a non-transitory computer readable memory including processor executable instructions that when executed by one or more processors perform the method of any of the above embodiments.

According to some embodiments, a system for characterizing movement kinematics is provided. The system includes at least one processor configured to: receive sensor data from one or more sensors indicating movement of the one or more body portions of the subject during movement of the subject; and use an exercise identification statistical model to determine a type of exercise during the movement of the subject based in part on the sensor data.

According to any of the above embodiments, the one or more sensors are attachable to one or more pieces of exercise equipment.

According to any of the above embodiments, the one or more sensors are wearable on one or more body portions of the subject.

According to any of the above embodiments, the one or more sensors include at least two sensors, and the sensor data includes at least first data and second data respectively received from the at least two sensors, wherein the first data and the second data are time-synchronized.

According to any of the above embodiments, the at least two sensors include a first sensor attached to a proximal portion of the subject and a second sensor attached to a distal portion of the subject.

According to any of the above embodiments, the proximal portion of the subject includes a torso, a thigh, or a proximal portion of a limb of the subject, and the distal portion of the subject includes an end of a limb of the subject.

According to any of the above embodiments, the sensor data includes at least a difference between a first reading from the first sensor and a second reading from the second sensor.

According to any of the above embodiments, the at least one processor is further configured to use the exercise identification statistical model to additionally determine at least a start point and/or a stop point during the movement of the subject.

According to any of the above embodiments, the sensor data includes at least one or more of acceleration, angular velocity, or magnetometer data.

According to any of the above embodiments, the exercise identification statistical model includes a dilated convolutional neural network.

According to some embodiments, a method for characterizing movement kinematics is provided. The method includes, by at least one processor: receiving sensor data from one or more sensors, the sensor data indicating movement of the one or more body portions of the subject during movement of the subject; and using an exercise identification statistical model to determine a type of exercise during the movement of the subject based in part on the sensor data.

According to any of the above embodiments, the one or more sensors are attachable to one or more pieces of exercise equipment.

According to any of the above embodiments, the one or more sensors are wearable on one or more body portions of the subject.

According to any of the above embodiments, the one or more sensors include at least two sensors, and the sensor data includes at least first data and second data respectively received from the at least two sensors, wherein the first data and the second data are time-synchronized.

According to any of the above embodiments, the at least two sensors include a first sensor attached to a proximal portion of the subject and a second sensor attached to a distal portion of the subject. According to any of the above embodiments, the proximal portion of the subject includes a torso, a thigh, or a proximal portion of a limb of the subject, and the distal portion of the subject includes an end of a limb of the subject.

According to any of the above embodiments, the sensor data includes at least a difference between a first reading from the first sensor and a second reading from the second sensor.

According to any of the above embodiments, the method further includes using the exercise identification statistical model to additionally determine at least a start point and/or a stop point during the movement of the subject.

According to any of the above embodiments, the sensor data includes at least one or more of acceleration, angular velocity, or magnetometer data.

According to any of the above embodiments, the exercise identification statistical model includes a dilated convolutional neural network.

According to some embodiments, a method for training a machine learning model is provided. The machine learning model is trained and configured to determine at least a type of exercise from a movement of a subject based on sensor data associated with the movement of the subject. The method of training includes, by at least one processor: receiving training sensor data collected at one or more sensors, the training sensor data indicating movement of one or more body portions of one or more subjects while performing one or more exercises; obtaining data indicating at least a type of exercise performed by the one or more subjects while the training sensor data is collected; and training the machine learning model to be configured to determine at least a type of exercise from a movement of a subject, by using the received training sensor data and the data indicating the at least a type of exercise associated with the training sensor data.

According to any of the above embodiments, the one or more sensors are attachable to one or more pieces of exercise equipment.

According to any of the above embodiments, the one or more sensors are wearable on one or more body portions of the one or more subjects.

According to any of the above embodiments, the one or more sensors include at least two sensors, and the training sensor data includes at least first training data and second training data respectively received from the at least two sensors, wherein the first training data and the second training data are time-synchronized.

According to any of the above embodiments, the training sensor data includes at least a difference between corresponding values in the first training data and the second training data.

According to any of the above embodiments, the method, further includes obtaining training start/stop data including one or more start points and/or one or more stop points in the training sensor data, wherein the one or more start points each indicates a start of an exercise when the training sensor data is collected, and wherein the one or more stop points each indicates a stop of an exercise when the training sensor data is collected, and training the machine learning model to additionally determine at least a start point and/or a stop point during the movement of the subject, by additionally using the training start/stop data.

According to any of the above embodiments, obtaining the training start/stop data includes timing a start and/or stop of an exercise performed by a respective subject of the one or more subjects when the training sensor data indicating movement of the respective subject is collected.

According to any of the above embodiments, the training sensor data includes at least one or more of acceleration, angular velocity, or magnetometer data.

According to any of the above embodiments, the machine learning model includes a dilated convolutional neural network.

According to some embodiments, a system for characterizing movement kinematics is provided. The system includes at least one processor configured to: receive sensor data from one or more sensors, the sensor data indicating movement of the one or more body portions during movement of the subject; and use a kinematic characteristic statistical model to determine one or more kinematic characteristics from the movement of the subject based on the sensor data.

According to any of the above embodiments, the one or more sensors are attachable to one or more pieces of exercise equipment.

According to any of the above embodiments, the one or more sensors are wearable on one or more body portions of the subject.

According to any of the above embodiments, the at least one processor is further configured to use an exercise identification statistical model to determine first data indicating at least a type of exercise during the movement of the subject based in part on the sensor data, and use the kinematic characteristic statistical model to determine the one or more kinematic characteristics from the movement of the subject based additionally on the first data from the exercise identification statistical model.

According to any of the above embodiments, the at least one processor is configured to determine the one or more kinematic characteristics from the movement of the subject by at least estimating movement velocity and trajectory from the sensor data.

According to any of the above embodiments, the at least one processor is configured to estimate the movement velocity and trajectory from the sensor data by estimating the movement velocity based on the sensor data, filtering the estimated movement velocity by removing low frequency bias, and estimating the movement trajectory based on the filtered movement velocity.

According to any of the above embodiments, the at least one processor is configured to filter the estimated movement velocity by using a movement kinematic machine learning model.

According to any of the above embodiments, the exercise identification statistical model and the movement kinematic machine learning model each include a respective dilated convolutional neural network.

According to any of the above embodiments, the at least one processor is further configured to use the exercise identification statistical model to determine second data indicating at least a start point and/or stop point during the movement of the subject based in part on the sensor data, and use the kinematic characteristic statistical model to determine the one or more kinematic characteristics from the movement of the subject additionally based on the second data.

According to any of the above embodiments, the sensor data includes at least one or more of acceleration, angular velocity, or magnetometer data.

According to any of the above embodiments, the one or more kinematic characteristics include one or more of a number of repetitions, a pace of repetitions, an overall consistency, a velocity, a trajectory, and/or a range of motion.

According to any of the above embodiments, the one or more sensors include a first sensor attached to a proximal portion of the subject and a second sensor attached to a distal portion of the subject.

According to any of the above embodiments, the proximal portion of the subject includes a torso, a thigh, or a proximal portion of a limb of the subject, and the distal portion of the subject includes an end of a limb of the subject.

According to any of the above embodiments, the sensor data includes at least a difference between a first reading from the first sensor and a second reading from the second sensor.

According to some embodiments, a method for characterizing movement kinematics is provided. The method includes, by at least one processor: receiving sensor data from one or more sensors, the sensor data indicating movement of the one or more body portions of the subject during movement of the subject; using a model to determine first data indicating at least a type of exercise during the movement of the subject based in part on the sensor data; and using a kinematic characteristic statistical model to determine one or more kinematic characteristics from the movement of the subject based on the sensor data.

According to any of the above embodiments, the one or more sensors are attachable to one or more pieces of exercise equipment.

According to any of the above embodiments, the one or more sensors are wearable on one or more body portions of the subject.

According to any of the above embodiments, the method further includes using an exercise identification statistical model to determine first data indicating at least a type of exercise during the movement of the subject based in part on the sensor data, and using the kinematic characteristic statistical model to determine the one or more kinematic characteristics from the movement of the subject based additionally on the first data from the exercise identification statistical model.

According to any of the above embodiments, determining the one or more kinematic characteristics from the movement of the subject includes at least estimating movement velocity and trajectory from the sensor data.

According to any of the above embodiments, estimating the movement velocity and trajectory from the sensor data includes estimating the movement velocity based on the sensor data, filtering the estimated movement velocity by removing low frequency bias, and estimating the movement trajectory based on the filtered movement velocity.

According to any of the above embodiments, filtering the estimated movement velocity includes using a movement kinematic machine learning model.

According to any of the above embodiments, the exercise identification statistical model and the movement kinematic machine learning model each include a respective dilated convolutional neural network.

According to any of the above embodiments, the method further includes using the exercise identification statistical model to determine second data indicating at least a start point and/or stop point during the movement of the subject based in part on the sensor data, and using the kinematic characteristic statistical model to determine the one or more kinematic characteristics from the movement of the subject additionally based on the second data.

According to any of the above embodiments, the sensor data includes at least one or more of acceleration, angular velocity, or magnetometer data.

According to any of the above embodiments, the one or more kinematic characteristics include one or more of a number of repetitions, a pace of repetitions, an overall consistency, a velocity, a trajectory, and/or a range of motion.

According to any of the above embodiments, the one or more sensors include a first sensor attached to a proximal portion of the subject and a second sensor attached to a distal portion of the subject.

According to any of the above embodiments, the proximal portion of the subject includes a torso, a thigh, or a proximal portion of a limb of the subject, and the distal portion of the subject includes an end of a limb of the subject.

According to any of the above embodiments, the sensor data includes at least a difference between a first reading from the first sensor and a second reading from the second sensor.

According to some embodiments, a method for training a machine learning model is provided. The machine learning model is trained and configured to determine one or more kinematic characteristics from a movement of a subject based on sensor data associated with the movement of the subject. The training method includes, by at least one processor: receiving training sensor data collected at one or more sensors, the training sensor data indicating movement of one or more body portions of one or more subjects during movement of the one or more subjects; obtaining first data indicating at least a type of exercise performed by the one or more subjects while the training sensor data is collected; tracking the movement of the one or more subjects while the training sensor data is collected to determine ground truth kinematic characteristics data; and training the machine learning model to be configured to determine one or more kinematic characteristics from a movement of a subject, by using the received training sensor data, the first data, and the ground truth kinematic characteristics data.

According to any of the above embodiments, the one or more sensors are attachable to one or more pieces of exercise equipment.

According to any of the above embodiments, the one or more sensors are wearable on one or more body portions of the one or more subjects.

According to any of the above embodiments, tracking the movement of the one or more subjects includes tracking movement of each of the one or more subjects by tracking movement of one or more markers placed on the subject during movement of the subject, and determining the ground truth kinematic characteristics data is based at least on the movement of the one or more markers for the one or more subjects.

According to any of the above embodiments, tracking the movement of the one or more subjects includes tracking movement of each of the one or more subjects by tracking positions and/or orientations of the one or more wearable sensors on the subject using at least two cameras during the movement of the subject, and determining the ground truth kinematic characteristics data is based on the positions of the one or more wearable sensors during the movement of the one or more subjects.

According to any of the above embodiments, tracking the movement of the one or more subjects includes tracking movement of each of the one or more subjects by receiving equipment movement data from a sensor installed on an exercise equipment on which the subject is exercising during the movement of the subject, and determining the ground truth kinematic characteristics data is based on the received data from the equipment movement data for the one or more subjects.

According to any of the above embodiments, the sensor installed on the exercise equipment includes a displacement sensor.

According to any of the above embodiments, the machine learning model includes a dilated convolutional neural network.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labelled in every drawing. In the drawings:

FIG. 1A is an illustrative diagram of a system for characterizing movements of a subject using an exercise identification statistical model, according to some embodiments;

FIG. 1B is an illustrative diagram of a system for characterizing movement kinematics of exercises using two statistical models, according to some embodiments;

FIG. 1C illustrates exemplary locations of sensors attachable to a subject or equipment for capturing sensor data indicating movement of one or more portions of the subject's body, according to some embodiments;

FIG. 2A is an illustrative diagram of an exemplary configuration of a kinematic characteristic statistical model for characterizing movement kinematics of exercises, according to some embodiments;

FIG. 2B is an illustrative diagram showing an exemplary implementation of an exercise identification statistical model and a movement kinematic machine learning model as shown in FIG. 1B, according to some embodiments;

FIGS. 3A-3B illustrate examples of different types of exercises and associated kinematic characteristics, according to some embodiments;

FIG. 4 is a flow diagram of an exemplary method for characterizing movement using an exercise identification statistical model, according to some embodiments;

FIG. 5 is a flow diagram of an exemplary method for characterizing movement kinematics of exercises using a kinematic characteristic statistical model, according to some embodiments;

FIG. 6A is a flow diagram of an exemplary method for training an exercise identification model, according to some embodiments;

FIGS. 6B-6C are flow diagrams of exemplary methods which can be used in a method for training an exercise identification model as shown in FIG. 6A, according to some embodiments;

FIG. 7 is a flow diagram of an exemplary method for training a movement kinematic machine learning model that can be part of a kinematic characteristic statistical model, according to some embodiments;

FIG. 8 is a diagram of an example combined model for kinematics estimation and activity classification, according to some embodiments of the technology described herein;

FIG. 9A is an example of a feature aggregation network for improving the accuracy of the activity identification and kinematic characteristics estimation of a model, according to some embodiments of the technology described herein;

FIG. 9B is an example of a feature aggregation network for improving the accuracy of estimation of kinematic characteristics, according to some aspects of the technology described herein;

FIG. 9C is an example of a feature aggregation network for improving the accuracy of activity identification, according to some aspects of the technology described herein;

FIG. 10 is an example process for training a combined activity identification and kinematics characteristics model, according to some aspects of the technology described herein;

FIG. 11A depicts a confusion matrix for the identification of strength training exercises during an experimental process, according to one embodiment;

FIG. 11B depicts an example of a trajectory for a biceps curl exercise during an experimental process, according to one embodiment;

FIG. 11C depicts a graph of trajectory error for different models determined from an experimental process, according to one embodiment;

FIG. 11D depicts a graph of velocity error for different models, determined from an experimental process, according to one embodiment;

FIG. 11E depicts a graph of trajectory errors across different exercises for different models, determined from an experimental process, according to one embodiment;

FIG. 11F depicts a graph of trajectory error for different models at different movement speeds, determined from an experimental process, according to one embodiment;

FIG. 11G depicts a graph of velocity error for different models at different movement speeds, determined from an experimental process, according to one embodiment;

FIG. 12A depicts a confusion matrix for the identification of activities during an experimental process, according to one embodiment;

FIG. 12B depicts a graph of results for joint angle error for different models determined during an experimental process, according to one embodiment;

FIG. 12C depicts a graph of angle error for different models across different activities, determined during an experimental process, according to one embodiment;

FIG. 12D depicts a graph of joint angle error for different models across different activities over time, determined during an experimental process, according to one embodiment; and

FIG. 13 shows an illustrative implementation of a computer system that may be used to perform any of the aspects of the techniques and embodiments disclosed herein, according to some embodiments.

### DETAILED DESCRIPTION

Quantified performance and assessments of exercises may provide information on quantified biomechanics which may precede obvious, functional changes of users. For example, quantified performance of exercises may include quantified movement kinematics, which may be used to evaluate, monitor and compare health states of a subject before and after a therapeutic or training session. Information on quantified performance can also be used to compare health states among a group of subjects under study in some examples.

The inventors have recognized and appreciated that existing systems for quantifying the kinematic characteristics using wearable technology mostly focus on movement and cardiovascular performance associated with aerobic activities or physical therapy, but do not assess other types of exercises or provide detailed assessment on particular activities, such as strength training and CrossFit activities. Further, existing gold standard devices for strength training assessment are restricted to clinic or lab settings and most machines can only support one exercise. These systems also often require a large number of sensors to be worn on a body of a subject. This increases the complexity of the system, including but not limited to, calibration of sensors, body dimension measurement, and complexity of software algorithms.

For example, conventional systems for monitoring and assessing movement of body parts of a subject may include optical camera-based motion capture systems. Such systems have been considered gold standard for measuring human partial or whole-body kinematics as they can provide sub-millimeter accuracy for movement trajectory estimation and sub-degree level accuracy for joint angles estimation. However, these camera-based systems are often limited by high cost, limited workspace, and visual occlusion issues.

To address the limitations of optical camera-based motion capture systems, inertial measurement units (IMUs) have been widely explored as a low-cost and wearable alternative for measuring human body movements. IMUs contain accelerometers, gyroscopes, and often magnetometers, which measure linear accelerations, angular velocities, and magnetic fields, respectively. Human limb positions and joint angles can be estimated from the accelerator and gyroscope measurements of IMUs. Despite being cheap, portable, and ubiquitous, IMUs have a wide range of kinematic tracking accuracy, typically an order of magnitude worse than that of the camera-based systems.

However, the accuracy of IMUs for kinematics estimation is often decreased by the presence of time-varying bias and noise in the raw linear acceleration and angular velocity measurements. From numerical integration, these biases and noise result in drift errors that accumulate over time. To address the IMU drift issue, existing approaches utilize kinematic constraints that are intrinsic to human anatomy to constrain drift accumulation, for example by using an IMU array with IMUs placed on all body segments or using IMUs placed on body segments with known constraints such as the knee and forearm. These approaches have demonstrated that kinematic constraints can effectively reduce IMU drift. However, the performance of these methods is dependent on the precision of body parameter measurements (e.g., limb length or joint ROM) and the formulation accuracy of the utilized kinematics constraint. Therefore, these methods are susceptible to error when the measurement of the constraints or the placement of the sensors is not accurate.

Conventional techniques have also tried to address IMU drift during walking tasks, where the numerical integration for each gait cycle is re-initialized during the point in the gait cycle when the foot is stationary relative to the ground. This approach, however, is limited only to cyclic activities where there are distinct periods where the user has zero velocity, and does not work well in activities where there is variation between cycles, such as upper body activities, which are less controlled than walking.

To solve the aforementioned technical problems and/or other technical problems, the inventors have recognized and appreciated that it may be advantageous to use a fewer number of sensors to quantify kinematic characteristics of exercises. As a result, the number of sensors and the complexity of the system can be decreased, and thus, the cost of the system can be reduced. Furthermore, the inventors have recognized and appreciated that it may be advantageous to develop systems and methods that can accurately identify movements associated with exercise versus non-exercise movements. Furthermore, the inventors have recognized and appreciated that it may be advantageous to develop systems and methods that can accurately quantify the kinematic characteristics associated with exercise of a subject.

As elaborated on below, the systems and methods disclosed herein to identify activities associated with certain movements of a subject and/or to quantify kinematic characteristics associated with such activities may be implemented and used in a variety of manners. This may include the separate training and use of separate models, separately trained models that are used together, and/or a single integrated model that is trained and used to identify both an activity and one or more associated kinematic characteristics. Embodiments related to each of these types of implementations is detailed further below.

In view of the foregoing, the inventors have developed new technologies for detecting exercises and characterizing movement kinematics of exercises. Described herein are various techniques, including systems and methods for characterizing movement kinematics of a subject using at least one processor, by: receiving sensor data from one or more sensors wearable on one or more body portions of a subject, and using an exercise identification statistical model to determine a type of exercise during the movement of the subject based in part on the sensor data. In some embodiments, the systems and methods may also include using the exercise identification statistical model to additionally determine at least a start point and/or a stop point during the movement of the subject.

In some embodiments, the sensor data may be collected from various sensors wearable on one or more body portions of the subject and indicate movement of the one or more body portions of the subject during movement of the subject. For example, a sensor may be an inertial measurement unit (IMU). Sensor data from IMU(s) may be used to produce various types of sensor data, such as acceleration (e.g., cartesian acceleration, rotational acceleration), angular velocity, and magnetometer data. Based on these sensor data, the system may obtain further measurements like velocity, trajectory, and, orientation (e.g., Euler angles, quaternions, and/or a rotation matrix). However, other appropriate types of sensors configured to measure any one or more of the above types of data may be used including, but not limited to, accelerometers, gyroscopes, barometers, heart beat sensors (e.g., ECG or any other kinds), temperature sensors, combinations thereof, and/or any other appropriate type of sensor.

Additionally, embodiments in which the one or more sensors include sensors that are configured to be disposed on one or more portions of exercise equipment that a subject interacts with to sense the desired motion data are also contemplated. For example, displacement sensors, accelerometers, gyroscopes, magnetometers, and/or any other appropriate sensor may be associated with one or more portions of an exercise equipment (e.g., a weight machine, treadmill, rowing machine, elliptical machine, or other type of exercise equipment) that are moved during usage and that may be used to characterize movements of a subject using the equipment. It should be understood that while a single sensor is discussed, any number of sensors (e.g., two, three, etc.) may be disposed on any appropriate moving and/or stationary portions of a piece of exercise equipment. These sensors may also optionally be used in combination with one or more wearable sensors as the disclosure is not limited to the number of location of the disclosed sensors.

In some examples, the type of exercise may indicate any suitable type of movement associated with an exercise, which will be further described in detail. In some embodiments, the sensed movement of a subject may include movements associated with both exercise and non-exercise motion. For example, a non-exercise motion may indicate the subject's movement that is not part of the exercise, such as getting water, walking between exercises, or any other movement that does not involve repetitions associated with the exercise to be identified.

The inventors have recognized and appreciated that conventional systems that employ wearable sensors often misclassify the exercise types due to similar motion patterns (e.g., push and pull motions) in different types of exercises. Further, the detection of start/stop points in an exercise in conventional systems may also be inaccurate because the sensor data may be insufficiently sensitive to the movement of the subject, causing the detection to occur too late or early.

In view of the above, in some embodiments as described further herein, the one or more sensors may include at least two sensors. For example, the at least two sensors may include different types of sensors and/or may be configured to capture different types of sensor data. In some embodiments, the at least two sensors may be worn on two different portions of a user's body. For example, a first sensor may be attached to a proximal portion of the user and a second sensor attached to a distal portion of the user. In non-limiting examples, the proximal portion of the subject comprises a torso, a thigh, an upper arm, or other proximal portion of a limb of the user. The distal portion of the subject may comprise a distal end portion of a limb of the user such as a calf, foot, forearm, hand, or other appropriate distal portion of a limb of a user. However, it should be understood that embodiments in which a single sensor configured to be worn on a single portion of a user's body is used to provide the disclosed sensor data associated motion of a portion of a subject's body may also be used as the disclosure is not so limited. Additionally, as noted above, in some embodiments equipment mounted sensors may also be used as the disclosure is not limited in this fashion.

In some embodiments, the sensor data may include first data and second data respectively received from the at least two sensors. In some embodiments, the first data associated with the first sensor and the second data associated with the second sensor are both captured at the same time instances such that the first data and the second data are time-synchronized. In some embodiments, the sensor data may include a difference of motion data between the first sensor and the second sensor. For example, the sensor data may include a plurality of data points, each representing an angular difference between corresponding values in the first data and the second data. In a non-limiting example, the difference of motion data may include Euler angles and/or acceleration.

It is appreciated that the use of two sensors attached to proximal and distal portions of a subject's body and/or attached to any suitable exercise equipment, and/or the use of difference of motion data between two sensors may increase the sensitivity of the statistical model to various types of exercises, and thus, improve the detection accuracy and robustness. For example, adding the quaternions of orientation differences between two sensors in addition to other sensor data (e.g., IMU data) may enable a statistical model to distinguish comparable linear motions but different forms of motions, such as bench press and shoulder press.

In some embodiments, the accuracy of the system may be further improved. For example, the exercise identification statistical model may include a convolutional neural network (CNN). In some embodiments, the exercise identification statistical model may include a dilated CNN, which is suitable for handling temporal sensor data as the data is collected sequentially during the movement of the user. In some embodiments, the exercise identification statistical model may include a stacked dilated CNN. However, it should be understood that any appropriate type of statistical model capable of being used to identify a desired type of exercise (e.g., Long Short-Term Memory (LSTM)) may be used as the disclosure is not so limited.

In view of the above, in some embodiments as described further herein, the exercise identification statistical model may be trained by using a method that includes receiving training sensor data collected at one or more sensors, the training sensor data indicating movement of one or more body portions of one or more subjects while performing one or more exercises. The training method may further include obtaining data indicating at least a type of exercise performed by the one or more subjects while the training sensor data is collected; and training the machine learning model to be configured to determine at least a type of exercise from a movement of a subject, by using the received training sensor data and the data indicating the at least a type of exercise associated with the training sensor data.

In some embodiments, the training sensor data may be collected from one or more wearable sensors, or sensors disposed on a moveable portion of an exercise equipment that a subject interacts with, in a similar manner as described above with respect to receiving the sensor data for performing inference on the exercise identification model. For example, the one or more wearable sensors for collecting training sensor data may include the same types of sensors (e.g., IMUs) and may be configured to provide the same types of sensor data. In some examples, at least two wearable sensors may be worn at different body portions of the subject. The training sensor data may include at least first training data and second training data respectively received from the at least two sensors, where the first training data and the second training data are time-synchronized. For example, the first training data may include a plurality of data points received from a first sensor, and the second training data may include a plurality of data points received from a second sensor. In some embodiments, the training sensor data may include a difference of motion data between corresponding values in the first training data and the second training data. It is appreciated that when using a trained exercise identification statistical model for identifying a type of exercise, the sensor data is collected in the same manner as the training sensor data is collected in training the exercise identification statistical model. In some embodiments, the trained exercise identification statistical model may include a dilated CNN. In some embodiments, the trained exercise identification statistical model may include a stacked dilated CNN. However, embodiments in which different types of statistical models are used and/or in which a different number of sensor data sets are used are also contemplated.

Described herein are various techniques, including systems and methods for characterizing movement kinematics of a subject performing an exercise using a kinematic characteristic statistical model. The systems and methods may include, using at least one processor: receiving sensor data from one or more sensors, and using a kinematic characteristic statistical model to determine one or more kinematic characteristics from the movement of the subject based on the sensor data. The one or more sensors may be attachable to one or more pieces of equipment with which the subject is performing the exercise. The one or more sensors may be wearable on one or more body portions of the subject. The sensor data may indicate movement of the one or more body portions during movement of the subject. For example, the sensor data may include at least one or more of: three axis acceleration data, angular acceleration data, magnetometer data, displacement data, and/or any other appropriate type of data that may be used to characterize movement of one or more portions of a subject's body. The number, type and location of sensors, and the collection of sensor data may be similar to those described above and further herein with respect to the exercise identification model. Additionally, in some embodiments, the sensor data may include a difference between a first reading from a first sensor and a second reading from a second sensor, though embodiments in which different numbers of sensors, including a single sensor, are used are also contemplated.

In any of the embodiments disclosed herein, the one or more kinematic characteristics may include one or more of: a number of repetitions, a pace of repetitions, an overall consistency, a (mean concentric) velocity, a trajectory, a range of motion, and/or any other appropriate type of kinematic characteristic.

In some embodiments, the kinematic characteristic statical model may be a regression model configured to generate regression output representing the one or more kinematic characteristics. In some embodiments, the kinematic characteristic statistical model may be configured to estimate movement velocity and trajectory from the sensor data, and use the estimated velocity and trajectory of the movement of the user to determine the kinematic characteristics. In some embodiments, the kinematic characteristic statistical model may use the estimated movement velocity and trajectory, and additionally characteristics of exercises to determine the one or more kinematic characteristics. For example, depending on the type of exercises, the kinematic characteristic statistical model may extract movement velocity and trajectory attributable to the sensor(s) that are sensitive to the type of exercise, and estimate the one or more kinematic characteristics based on the extracted movement velocity and trajectory. In some embodiments, the characteristics of exercises (e.g., type of exercises) may be obtained from the exercise identification statistical model, such as what is described above and further herein.

The inventors have recognized and appreciated that IMU-based velocity and trajectory estimation may not be accurate in conventional systems. For example, IMU acceleration measurements may include errors due to time-varying biases and random noise in IMU sensor data. Over time, the error from biases may accumulate to introduce IMU drifts. To overcome this, in commercial systems, a large number of sensors may be worn on a body to reduce the drift errors. However, this is not practical. Accordingly, the inventors have developed techniques to enable reliable use of sensor data with a fewer number of sensors and provide accurate estimate of the kinematic characteristics, as will be described further herein.

In some embodiments, movement velocity and trajectory may be estimated using the acceleration measurements in the sensor data. In reducing sensor drift in the sensor data, the kinematic characteristic statistical model may be configured to filter the estimated movement velocity by removing low frequency bias and estimating the movement trajectory based on the filtered movement velocity. In some examples, filtering the estimated movement velocity may be performed using a high-pass filter to remove motion data below a threshold frequency to eliminate the low frequency bias component in the movement velocity. In some examples, filtering the estimated movement velocity may be performed using a machine learning model (or other appropriate statistical model) to learn the behaviour of bias drifts so that the real signal without biases can be accurately estimated.

In some embodiments, the machine learning model in the kinematic characteristic statistical model can be trained to also learn the characteristics of exercises, and additionally use the characteristics of exercises to estimate the movement velocity and trajectory. In some embodiments, the characteristics of exercises may include a type of exercise, which may be obtained from the exercise identification statistical model as described above and further herein, using the sensor data. The characteristics of exercises may also include start/stop points during the exercises, which may also be obtained from the exercise identification statistical model as described above and further herein, using the sensor data. The characteristics of exercises may be features used for training the machine learning model. In a non-limiting example, the machine learning model in the kinematic characteristic statistical model may include a CNN, such as a dilated CNN, a stacked dilated CNN, similar to the exercise identification model. Other appropriate statistical models may also be used as the disclosure is not so limited.

In view of the above, in some embodiments as described further herein, the machine learning model in the kinematic characteristic statistical model may be trained by using a method that includes receiving training sensor data collected at one or more sensors, tracking the movement of the one or more subjects while the training sensor data is collected to determine ground truth kinematic characteristics data, and training the movement kinematic machine learning model by using the received training sensor data, and the ground truth kinematic characteristics data. In some embodiments, the machine learning model may include a CNN, such as a dilated CNN, a stacked dilated CNN, or other appropriate statistical model similar to the exercise identification model detailed above.

As noted above, using kinematic characteristics in activity identification and using activity type in kinematic characteristic determination may improve the accuracy of both. Accordingly, the Inventors have recognized the benefits associated with an integrated statistical model capable of performing both activity identification and kinematic characterization determination. For example, in some embodiments, the outputs of one or more layers of a statistical model associated with determining the kinematic characteristics or the activity identification may be passed to one or more layers associated with the other active step. For example, the output of a layer associated with determining the one or more kinematic characteristics may be passed to one or more layers of the statistical model associated with activity identification. Similarly, the output of a layer associated with activity identification may be passed to the one or more layers of the statistical model associated with determining the one or more kinematic characteristics.

In view of the above, the inventors have recognized the benefits associated with embodiments related to systems and methods for identifying an activity and characterizing movement kinematics of a subject using a combined activity identification and kinematic characteristics statistical model. The systems and methods may include, using at least one processor: receiving sensor data from one or more sensors, and using a combined activity identification and kinematic characteristics statistical model to determine an activity type and one or more kinematic characteristics from the movement of the subject based on the sensor data. The one or more sensors may be attachable to one or more pieces of equipment with which the subject is performing the activity and/or the one or more sensors may be wearable on one or more body portions of the subject. The sensor data may indicate movement of the one or more body portions during movement of the subject. For example, the sensor data may include at least one or more of: three axis acceleration data, one or more sets of single axis acceleration data, angular acceleration data, magnetometer data, displacement data, combinations of the forgoing, and/or any other appropriate type of data that may be used to characterize movement of one or more portions of a subject's body. The number, type and location of sensors, and the collection of sensor data may be similar to those described above and further herein with respect to any of the other models described herein. Additionally, in some embodiments, the sensor data may include a difference between a first reading from a first sensor and a second reading from a second sensor, though embodiments in which different numbers of sensors, including a single sensor, are used are also contemplated.

It should be understood that a combined model may be implemented in a number of different ways. For example, in some embodiments, a combined exercise identification and kinematic characteristics model may be implemented as separate exercise identification and kinematic characteristics models which are interconnected. In other embodiments, a combined exercise identification and kinematic characteristics model may be implemented as a single multilayer model with feedbacks between the different layers or sections corresponding to the exercise identification statistical model and kinematic characteristic statistical model. Additionally, in some examples, information may be processed as it is passed between the exercise identification model and the kinematic characteristics model. For example, the information may be filtered, processed by one or more model layers, or undergo other processing.

In one embodiment of a combined model, the exercise identification and kinematic characteristics model may include stacked dilated convolutional neural networks (DCNNs), and the final hidden layer of stacks of one, or both, of the models may be passed to stacks of the other model. For example, the final hidden layer of each stack of the exercise identification model may be passed to each stack of the kinematic characteristics model. In some examples, the final hidden layer of each stack of the kinematic characteristics model may be passed to each stack of the exercise identification model.

The exercise identification and kinematic characteristic models or sections of a combined model may be structured and trained, as described herein. For example, the combined exercise identification and kinematic characteristics statistical model may include a convolutional neural network (CNN). In some embodiments, the combined model may include a dilated CNN, which is suitable for handling temporal sensor data as the data is collected sequentially during the movement of the user. In some embodiments, the combined model may include a stacked dilated CNN. However, it should be understood that any appropriate type of statistical model capable of being used in the combined model (e.g., Long Short-Term Memory (LSTM)) may be used as the disclosure is not so limited.

In view of the above, in some embodiments as described further herein, the combined model may be trained by using a method that includes receiving training sensor data collected by one or more sensors, the training sensor data indicating movement of one or more body portions of one or more subjects while performing one or more exercises. The training method may further include obtaining data indicating at least a type of exercise and one or more kinematic characteristics performed by the one or more subjects while the training sensor data is collected; and training the machine learning model to be configured to determine at least a type of exercise and one or more kinematic characteristics from a movement of a subject, by using the received training sensor data and the data indicating the at least a type of exercise and one or more kinematic characteristics associated with the training sensor data.

As noted above, a combined model may be implemented in a number of ways. In either case, the inventors have recognized and appreciated that sharing information between an exercise identification model and a kinematic characteristics model whether implemented as separate models and/or as a single integrated model may improve the accuracy of the identification of the exercises and kinematic characteristics of the movement of the subject. For example, information from the identification statistical model may be passed to the kinematic characteristic statistical model, which improves the accuracy of the estimated kinematic characteristics. Similarly, information from the kinematic characteristic statistical model may be passed to the exercise identification statistical model, which improves the accuracy of the identified exercises. Sharing may also occur in both directions. For instance, information may be shared from the exercise identification model to the kinematics estimation model and from the kinematics estimation model to the exercise identification model. That said, in some examples, information may only be shared in a single direction, for example from the kinematic characteristics model to the exercise identification model or from the exercise identification model to the kinematic characteristics model. Such sharing of information may lead to improved accuracy and may be implemented in a number of ways as elaborated on further below.

For the various embodiments disclosed herein, the training sensor data may indicate movement of one or more body portions of one or more subjects during movement of the one or more subjects. The training sensor data may be collected in a similar manner (with respect to, for example, the type of sensor data, the number, type and location of sensors etc.) as the training sensor data is collected for training the exercise identification statistical model and kinematic characteristics statistical model.

In some embodiments, in determining the ground truth kinematic characteristics data, such as movement velocity and trajectory, the training method may track the movement of the subjects performing the exercises. In some embodiments, tracking the movement of the subject(s) may include a marker-based approach, which uses a motion capture system to track the positions of one or more markers placed on the body of the subject(s), and uses the tracked positions of the marker(s) to estimate the ground truth kinematic characteristics data (e.g., ground truth movement velocity and trajectory)

Alternatively, and/or additionally, tracking the movement of the subjects may include using one or more cameras to track the positions and/or orientations of the one or more wearable sensors attached to the body of the subject(s). In some embodiments, such sensors may correspond to one or more motion tracking markers attached to the subject's body which may be monitored using one or more cameras or other type of photosensitive detector capable of imaging and tracking movement of the markers within a field of view of the system. Subsequently, the training method may use the tracked positions and/or orientations of the sensor(s) to determine the ground truth kinematic characteristics data.

Alternatively, and/or additionally, tracking the movement of the subjects may include tracking the movement of each of the one or more subjects by receiving equipment movement data from a sensor installed on an exercise equipment on which the subject is exercising during the movement of the subject; and estimating the ground truth kinematic characteristics data based on the received data from the equipment movement data for the one or more subjects. For example, tracking the movement may include receiving sensor data from a displacement sensor such as a linear variable differential transformer (LVDT), encoder, or other appropriate sensor installed on suitable portion of equipment, e.g., moveable portion of a bike, a treadmill, or other exercise equipment that moves (or vibrates, or otherwise respond with other measurable readings) during operation by a subject performing a desired exercise.

The various embodiments described above and further herein provide advantages over conventional systems in identifying types of exercise and/or characterizing kinematic characteristics of exercises. For example, the various embodiments in the present disclosure may use fewer sensors (e.g., one, two, or more sensors) wearable at different body portions of a user and/or attachable on equipment, and one or more statistical models to accurately estimate the kinematic characteristics of exercises.

In some embodiments, and as described above and further herein, benefits related to the use of exercise identification information in kinematics estimation may include increased accuracy of the kinematics estimations with a lower number of sensors and less computationally expensive processing than conventional techniques. In contrast, conventional techniques for improving accuracy of kinematic characteristics estimations and exercise identification have incorporated additional sensors across the entire body of a subject and/or incorporated additional sensing modalities. The increased number of sensors associated with such techniques increases the complexity and cost of systems and require additional computing power to process the increased data as compared to the currently disclosed systems and methods.

The systems and described herein may reduce or eliminate the use of additional sensing modalities, such as additional sensors beyond IMU sensors in some embodiments, for accurate exercise identification and kinematic characteristics estimation. Again this use of both few numbers and fewer types of sensors sensing different locations and types of data may reduce the number of sensors and the associated computational cost while provide more accurate estimations.

The systems and techniques described herein may also provide reduced long-term drift of IMU measurements. Specifically, passing information between the exercise identification model and the kinematic characteristics model may reduce IMU drift error during use while providing improved data and estimation over conventional techniques with fewer sensors and less processing than conventional techniques.

The systems and described herein may additionally provide accurate estimations of user exercise identity and kinematic characteristics across multiple subjects, without the need for subject-specific calibration. Conventional techniques often require subject-specific calibration for accurate analysis, which may limit their applicability and efficiency in use for a broad population.

In some embodiments, as described above and further herein, the systems and techniques may be used in strength training and rehabilitation applications. For example, different exercises and/or activities may be determined from data measured during a subject's training. In some examples, kinematic characteristics may be determined during a subject's training, for example range of motion, mean velocity, and peak velocity. In some examples, kinematic characteristics determined during training may be used in determining one or more user attributes such as injury risk, muscle development, muscle strength, neuromuscular fatigue, joint kinematics, joint angle, changes in metrics overtime, evaluation of a subject's performance of an activity, and/or any other desired metric.

As noted above, the methods and systems disclosed herein may be used to identify different types of activities and associated kinematic characteristics. Accordingly, it may also be desirable for a system to be able to distinguish between multiple different types of activities using a single model. Thus, an activity identification model, or section of a combined model, may be configured to determine a type of activity of a subject from a predetermined list of activities that the system has been trained to identify. Such a list of activities may correspond to any of the types of activities disclosed herein. For example, in one embodiment, the different activities may be different types of exercises, though implementations in which other types of non-exercise activities are included are also contemplated. Similar to the above, a kinematic characteristic statistical model may be configured to determine one or more kinematic characteristics associated with the different types of activities. Again, such functionality may either be implemented using separate models and/or using a combined model as the disclosure is not so limited.

In some embodiments, as described above and further herein, the systems and techniques disclosed herein may be used in analysing user activities other than exercises. For example, the activity identity and kinematics may be estimated during industrial tasks. In some examples, joint kinematics, joint angle, shoulder elevation, hand positioning, tool positioning, and/or posture may be analyzed from data recorded during overhead industrial work. In some examples, the estimated identity and kinematics may be used in ergonomics applications, such as risk assessment and injury prevention.

In some examples, the techniques described herein may be incorporated into wearable assistive robots, garments (e.g., sleeves, shorts, pants, leggings, socks, shoes, shirts, etc.), wearable structures (e.g., bracelets, necklaces, cuffs, watches, etc.), and/or any other embodiment where the sensors may worn on a subject's body. In come embodiments, the associated one or more processors may also be incorporated into any of the above noted wearable systems.

In embodiments as described above and further herein, examples of exercises may include any types of desired physical movement of a subject to be identified and/or quantified. For example, an exercise may include repetitive movement of body parts. An exercise can be performed in any suitable settings, such as gyms, clinics, home, nature park etc. In non-limiting embodiments, the type of exercises may include any suitable exercises that require movement of one or more body portions, for example, exercises performed in strength training, or other exercises. In non-limiting examples, the type of exercises may include one or more of: push-ups, biceps curls, tricep extensions, lateral raises, shoulder extensions, rear flys, contra-stabilized latissimus-dorsi pulldowns, chest presses, contra-stabilized rows, contra-stabilized rear flys, overhead presses, low rows, high rows, shoulder flexions, plantarflexions, single-leg plantarflexions, squats, single-leg squats, split lunges, rear lunges, forward lunges, side lunges, monster walks, hip abductions, hamstring curls, hip extensions, hip flexions, knee extensions, deadlifts, single-leg deadlifts, hip bridges, single-leg bridges, Superman extensions, quadruped contralaterals, quadruped unilaterals, swimming, Russian twists, dead-bug contralaterals, dead-bug unilaterals, side planks on knees, side planks with one leg straight, side planks with straight leg and staggered feet, and side planks with straight legs stacked.

It should be understood that any of the methods and systems disclosed herein may be used to classify and determine kinematic characteristics for any type of activity. Accordingly, as described herein, all embodiments related to and/or references made to an exercise, an exercise type, an exercise identification statistical model, and/or other similar terms should be understood to also refer generally to other types of activities as well as the use and implementation of statistical models that may be used to identify and/or evaluate any appropriate type of activity and/or kinematic characteristics associated with such activities. Accordingly, in addition to the disclosed types of exercises described both above and elsewhere herein, types of activities that may be identified and/or evaluated for kinematic characteristics may include, but are not limited to: exercises, rehabilitation activities, walking, cleaning (e.g., washing dishes, vacuuming, etc.), industrial work (e.g., lifting, hammering, drilling, carrying loads, etc.), and/or any other type of activity involving movement of a subject which may be identified and/or characterized using the systems and methods disclosed herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

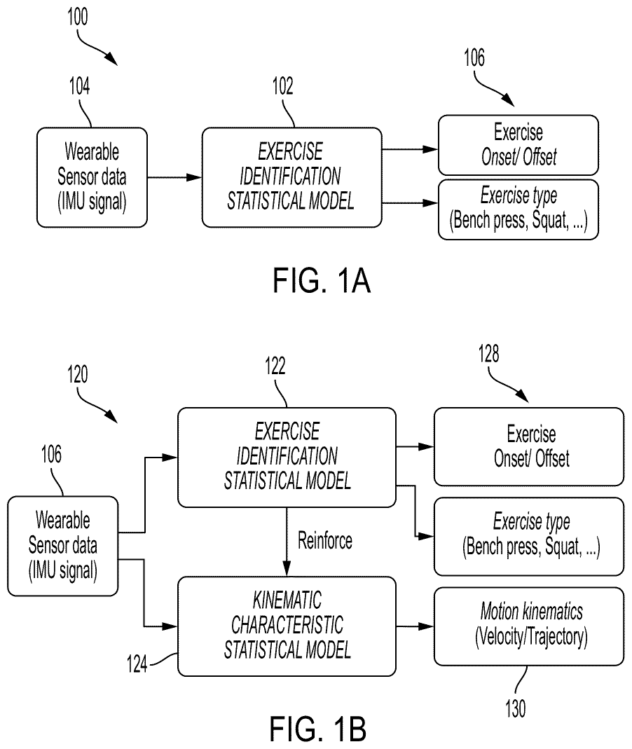

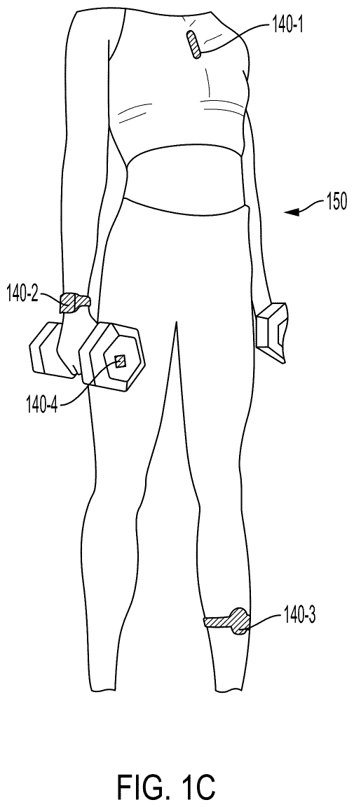

FIG. 1A is an illustrative diagram of a system 100 for characterizing movement of a subject using an exercise identification statistical model 102, according to some embodiments. The exercise identification statistical model 102 and the training thereof may be similar to what is described above and further herein. For example, the exercise identification model 102 may include a CNN, such as a dilated CNN, or a stacked dilated CNN, or any other appropriate statistical model. The exercise identification statistical model may be provided with sensor data as input, and may provide characteristics of exercises 106 as output. The sensor data 104 may be received from one or more sensors attached to one or more body portions of a subject or exercise equipment, where the sensor data indicates the movement of the one or more body portions of the subject during movement of the subject. The sensor data may be collected in a similar manner (with respect to, for example, the type of sensor data, the number, the type and the locations of sensors etc.) as what is described above for the exercise identification statistical model. It should be understood that receiving the data and providing the data to the statistical model from the one or more sensors may either be done in real time, or the data may be recalled from non-transitory computer readable memory, as the disclosure is not limited to when or how the data is analysed. The characteristics of exercises 106 may include the type of exercise being performed by the subject, and/or start/stop points in the sensor data.

FIG. 1B is an illustrative diagram of a system 120 for characterizing movement kinematics of exercises using two statistical models, according to some embodiments. As shown in FIG. 1B, system 120 includes an exercise identification statistical model 122 and a kinematic characteristic statistical model 124, the descriptions of which are provided above and further herein. System 120 is similar to system 100 with the difference being that a second statistical model, e.g., kinematic characteristic statistical model 124 is provided to additionally determine one or more kinematic characteristics 130 as described above and further herein. For example, the one or more kinematic characteristics 130 may include number of repetitions, pace of repetitions and overall consistency, and for each repetition, performance data such as velocity of movement, range of motion, and trajectory of sensors.

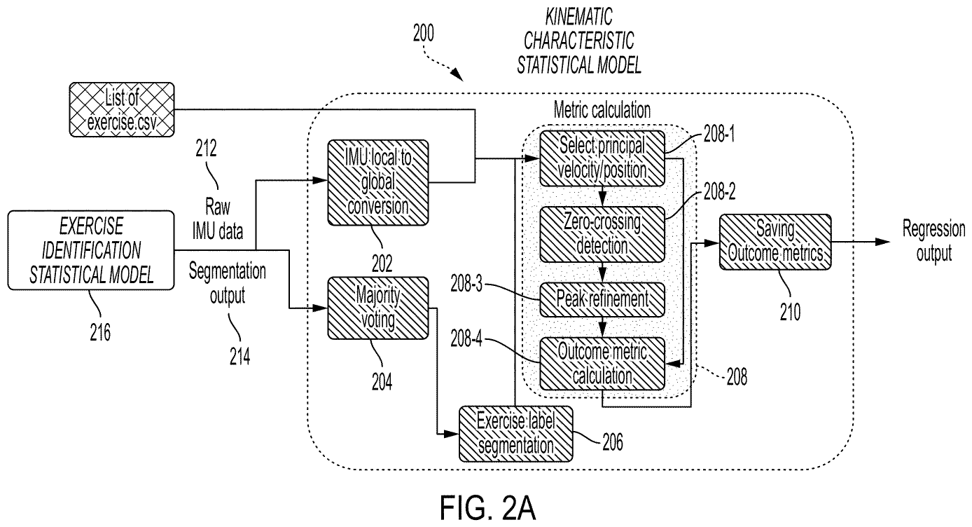

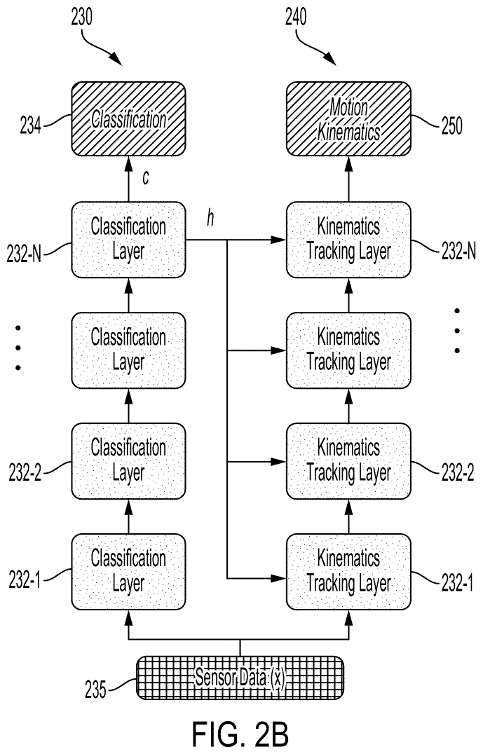

In some embodiments, the kinematic characteristic statistical model 124 and the exercise identification statistical model 122 are provided with the same sensor data 126. In some embodiments, sensor data 126 is collected in a similar manner as sensor data 104 is collected (FIG. 1A). In some embodiments, information from the exercise identification statistical model 122 may be used to reinforce the operation/learning of the kinematic characteristic statistical model, which is further described in detail with reference to FIGS. 2A-2B.

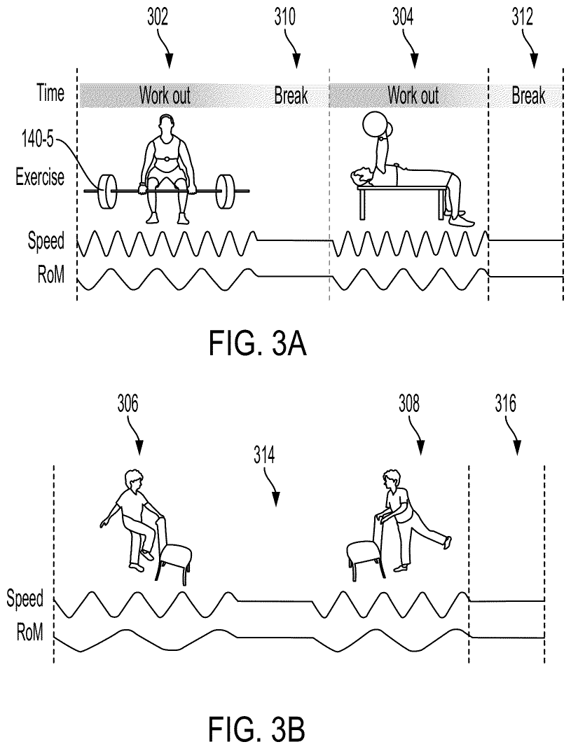

FIG. 1C illustrates exemplary locations of sensors attachable to a subject 150 or equipment for capturing sensor data indicating movement of one or more portions of the subject's body, according to some embodiments. One or more sensors (e.g., 140-1, 140-2, 140-3 etc.) may be attachable to various body portions of the subject during movement of the subject. One or more sensors (e.g., 140-4, 140-5 in FIG. 3A) may be attachable to a piece of equipment (e.g., weight(s), a bike, a treadmill etc.). In some embodiments, the sensors 140s may be used with any of the systems 100 (FIG. 1A), 120 (FIG. 1B) to provide the sensor data. As shown in FIG. 1C, the selection of sensors may depend on the type of exercises to characterize. For example, one sensor 140-1 may be sufficient to characterize movement kinematics of a push-up exercise, where sensor 140-3 may be used to characterize movement kinematics of leg exercises. It is appreciated that one, two, or more sensor may be used. Additionally, embodiments in which the one or more sensors are disposed on a portion of an exercise equipment that moves during operation by a subject for characterizing movement of a portion of the subject's body are also contemplated as detailed previously above.

FIG. 2A is an illustrative diagram of an exemplary configuration of a kinematic characteristic statistical model 200 for characterizing movement kinematics of exercises, according to some embodiments. In some embodiments, kinematic characteristic statistical model 200 may be implemented in kinematic characteristic statistical model 124 (FIG. 1B). In some embodiments, kinematic characteristic statistical model 200 may include one or more components (e.g., 202-210) configured to receive the sensor data 212 and segmentation output 214 as input, and provide the regression output (e.g., one or more kinematic characteristics, e.g., 130 in FIG. 1B).

With further reference to FIG. 2A, sensor data 212 may be received from one or more sensors (e.g., sensor data 126 as described in FIG. 1B) as described above and further herein. Sensor data 126 may include IMU data (e.g., acceleration, angular velocity and quaternion) or data from any other appropriate type of sensor wearable on any portion of a subject and/or attachable to any suitable exercise equipment. Segmentation output 214 may be provided from exercise identification statistical model 216. In some embodiments, exercise identification statistical model 216 may be implemented in exercise identification statistical model 102 (FIG. 1A), 122 (FIG. 1B). In some embodiments, the segmentation outputs 214 may include a sequence of data points in a single array comprising estimated exercise types at each timestamp.

With further reference to FIG. 2A, the input data 212, 214 may be provided to components 202, 204 of the statistical model 200. In some embodiments, a majority voting component 204 may be configured to smooth out the output from the exercise identification statistical model 216, where the output may be fragmented. In some embodiments, majority voting component 204 may be configured to receive a predefined majority voting window size (for example, 150 samples, or any other suitable number) and a voting threshold (for example, 20%, or any other suitable number). The majority voting component 204 may be configured to further determine the major type of exercise within the specified window by utilizing a sliding window (e.g., swiping the entire time series at a stride with a specified window). The majority voting component 204 may be configured to further determine a major element corresponding to a respective type of exercise. For example, if the number of major element exceeds a particular percentage, or other appropriate threshold, of the total window size, component 204 may update the output with the major element. Otherwise, the output may be marked as 0. (i.e. non-exercise label).

With further reference to FIG. 2A, the kinematic characteristic statistical model 200 may further include a segmentation component 206. The segmentation component 206 may receive the majority type of exercise from the majority voting component 204 and group individual exercises from consecutive exercise/non-exercise labels from the majority voting component 204. The segmentation component 206 may extract the indices of start and end points and timestamps of each exercise label from the entire exercise/non-exercise labels from the majority voting algorithm. In some examples, the segmentation component 206 may save the extracted information in exercise segments. In some embodiments, the exercise segments may be saved in a computer readable format and/or human readable format, e.g., in python dictionary format, or any other suitable format for the next operation. Examples of exercise segments are shown in FIGS. 3A-3B.

FIGS. 3A-3B illustrate examples of different types of exercises and associated kinematic characteristics, according to some embodiments. As shown, each of the segments 302, 304, 306, 308 may represent an exercise of a respective type, and segments 310, 312, 314, 316 may represent a non-exercise segment.

Returning to FIG. 2A, additionally and/or alternatively, the kinematic characteristic statistical model 200 may further include an IMU, or other sensor, conversion component 202 to estimate movement velocity and trajectory from the raw IMU data. The conversion component 202 may first convert the coordinates of raw sensor data (local coordinates) to a global coordinates (global frame). Various techniques for converting local sensor data to global frame based sensor data are available. For example, global accelerations can be calculated by multiplying rotation matrix obtained from IMU with local accelerations.

The IMU conversion component 202 may integrate the global frame based sensor data (e.g., acceleration) to estimate motion characteristics (e.g., movement velocity). Further, the IMU conversion component 202 may be configured to filter low frequency bias component in the movement velocity, for example, by using a high-pass filter that reduces, or eliminates, components of the data below a threshold frequency which may help to eliminate the low frequency bias component in the movement velocity. In some embodiments, a kinematic characteristic statistical model may be trained to learn the pattern of biases and used to estimate the movement velocity. Further, the IMU conversion component 202 may be configured to use the filtered movement velocity as described above to estimate movement trajectory.

With further reference to FIG. 2A, the kinematic characteristic statistical model 200 may further include a metric calculation component 208 to generate the regression output (e.g., one or more kinematic characteristics, such as, number of repetitions, mean concentric velocity, etc.) from each exercise identified by the exercise identification statistical model 216. In some embodiments, component 208 may include multiple sub-components, e.g., 208-1 . . . 208-4 (or any suitable number of sub-components). For example, a principal selector 208-1 may be configured to extract the primary movement velocity and trajectory based on the type of exercise identified from the exercise identification statistical model 216. In some embodiments, given an exercise segment, principal selector 208-1 may determine the primary sensor location (e.g., wrist IMU) that corresponds to the type of exercise, and extract corresponding movement velocity and trajectory attributable to the primary sensor.

Additionally, and/or alternatively, the principal selector 208-1 may determine the principal velocity and trajectory based on the direction of the motion. For example, if the exercise segment includes primarily horizontal plane motion (e.g., Erg Rowing), the principal selector 208-1 may select X and Y plane movement velocity and trajectory as the primary movement velocity and trajectory. In some embodiments, the principal selector 208-1 may employ Principal Component Analysis to determine the principal velocity and trajectory. Otherwise, the movement velocity and trajectory in Z-axis (which is parallel to gravity) may be selected as the principal movement velocity and trajectory. In some embodiments, if the exercise segment falls into the predefined category of dynamic exercise (e.g. jumping jacks), which generates a lot of high frequency noise out of the true signal, the principal selector 208-1 may apply a low pass filter on the principal movement to remove the noise.

With further reference to FIG. 2A, zero-crossing detector 208-2 may be configured to identify the indices of zero velocity points from the principal movement velocity and trajectory from the principal selector 208-1. In some embodiments, the zero-crossing detector 208-2 may differentiate between the zero velocity point of negative gradient and positive gradient, and identify the indices of positive and negative peaks of movement trajectory. In some examples, if the distance between two neighbouring zero velocity points is shorter than a threshold (e.g. 20 samples, or any other suitable numbers), the two neighbouring zero velocity points may be cancelled out, assuming they do not relate to actual exercise motion.

With further reference to FIG. 2A, peak selector 208-3 may be configured to identify positive and negative peaks of movement trajectory (via zero velocity points) in order to determine the number of exercise repetitions. In some embodiments, if two continuous positive or negative movement peaks are detected, peak selector 208-3 may cancel out those peaks, assuming that exercise motion must be cyclic (e.g. flexion and extension, or push and pull).

With further reference to FIG. 2A, metric calculator 208-4 may be configured to calculate the output one or more kinematic characteristics (e.g. number of repetitions, mean concentric velocity, etc.) based on the detected movement peaks from the peak selector 208-3 and the principal movement velocity and trajectory. In some examples, if the absolute distance between two positive and negative movement trajectory peaks exceeds a threshold (e.g., 2 meters or other suitable number), the pair of positive and negative peaks may be skipped, presuming that movement displacement cannot exceed a certain threshold. The output one or more kinematic characteristics may be saved to a computer readable and/or human readable format, e.g., in a set (i.e. a group of repetitions) in python list format.

With further reference to FIG. 2A, an output component 210 in the kinematic characteristic statistical model 200 may be configured to sort out the output metrics by the number of sets and the name of exercises, and save them in a computer readable and/or human readable format, e.g., Json format.

Although the kinematic characteristic statistical model 200 is described by various components thereof, e.g., 202-210, it is appreciated that fewer or more components may be possible. For example, any component may be integrated into another component such that a fewer component is used.

In some variations, kinematic characteristic statistical model 200 may include additional components to process any of the intermediate results from any components described above. For example, a component may be disposed between the majority voting component 204 and segmentation component 206 to find repetition errors, where a repetition is missed or a wrong repetition is added (e.g. when someone peaks up a weight). In finding the repetition errors, the component may be configured to look at the pattern of repetitions within the data set labelled as an exercise and then search for a similar data outside of the set. If similar data is found outside of the data set, then the component may adjust the boundaries of the data set labelled as an exercise. Additionally, the component may also validate that all the repetitions detected within a data set have similar patterns, where non-similar patterns may be attributed to accidental repetitions, and thus, may be removed. In some embodiments, metric calculation component 208 may also be configured to detect individual repetitions. In some examples, after the type of exercise, or other activity, is determined and the kinematic characteristics are determined, the type of exercise, or other activity, may be redetermined by exercise identification statistical model 216 using the kinematic characteristics. Alternatively, the type of activity and the one or more kinematic characteristics may be determined simultaneously as disclosed elsewhere herein.

FIG. 2B is an illustrative diagram showing an exemplary implementation of an exercise identification statistical model 230 and a movement kinematic machine learning model 240. In some examples, the movement kinematic machine learning model 240 may be part of a kinematic characteristic statistical model, such as 124 (FIG. 1B) and 200 (FIG. 2A). As described above and further herein, the movement kinematic machine learning model 240 may be configured to estimate the movement velocity and trajectory. In some embodiments, the exercise identification statistical model 230 may be implemented in exercise identification statistical model 102 (FIG. 1A), 122 (FIG. 1B).

In some embodiments, as shown in FIG. 2B, both models 230, 240 may be provided with sensor data 235 as input. In some embodiments, sensor data 235 may be collected and received in the manner describe above and further herein, such as sensor data 104 (FIG. 1A), 106 (FIG. 1B). Model 230 may be configured to provide the classification results (e.g., type of exercise, and/or start/stop points), and model 240 may be configured to provide one or more kinematic characteristics, as describe above and further herein. Each of the exercise identification statistical model 230 and movement kinematic machine learning model 240 may be a stacked neural network and may include multiple network layers. For example, exercise identification statistical model 230 may include multiple layers 232-1, 232-2, . . . , 232-N. Similarly, movement kinematic machine learning model 240 may include multiple layers 242-1, 242-2, . . . , 242-M.

In a non-limiting example, models 230, 240 may each use a stacked convolution structure. In some embodiments, in reinforcing the output of the exercise identification statistical model 230 into the learning of the movement kinematic machine learning model 240, feature propagation may be used through summation. For example, the processed feature of the final hidden layer of the exercise identification statistical model 230 (e.g., layer 232-N), h, is summed with the output of each layer in the movement kinematic machine learning model 240 and provided to the next layer. The output of the final layer is trajectory and velocity for every time t.

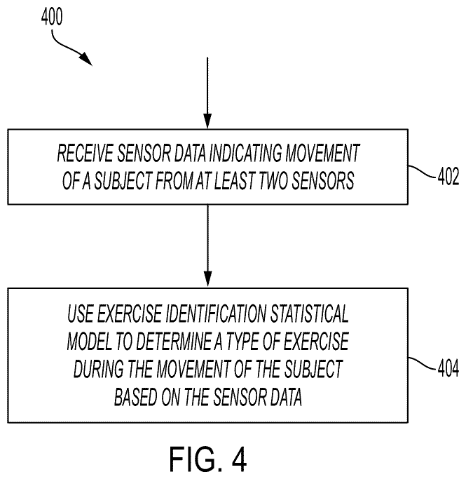

FIG. 4 is a flow diagram of an exemplary method 400 for characterizing movement using an exercise identification statistical model, according to some embodiments. In some embodiments, method 400 may include receiving sensor data from one or more sensors wearable on one or more body portions of a subject and/or configured to be disposed on a portion (e.g., moving portion) of a piece of exercise equipment, at act 402. The sensor data may be collected from one or more sensors in a similar manner as described above with respect to the exercise identification model. Similarly, the number, type and location of sensors may be similar to those described above and further herein with respect to the exercise identification model.

With further reference to FIG. 4, method 400 may further include using an exercise identification statistical model to determine a type of exercise during the movement of the subject based in part on the sensor data, at act 404. In some embodiments, the method may also include using the exercise identification statistical model to additionally determine at least a start point and/or a stop point during the movement of the subject. In some embodiments, method 400 may be implemented in the exercise identification statistical model, e.g., 102 (FIG. 1A), 122 (FIG. 1B), 216 (FIG. 2A), 230 (FIG. 2B). Thus, the acts in method 400 in the exercise identification statistical model may be similar to those described in the various embodiments in FIGS. 1A, 1B, 2A, and 2B.

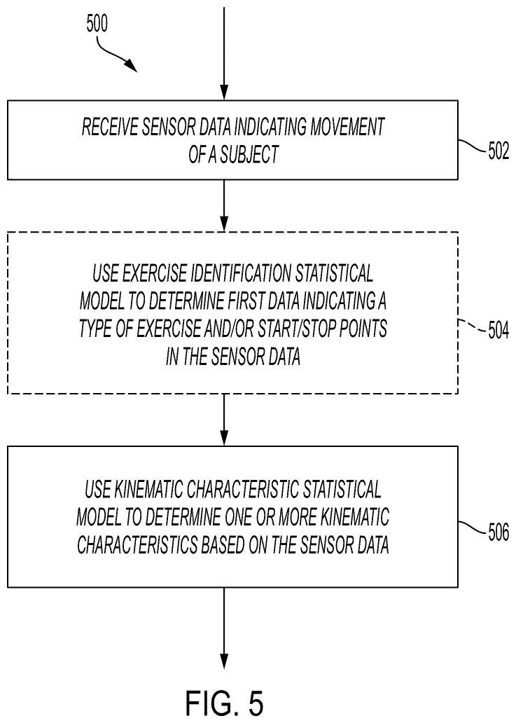

FIG. 5 is a flow diagram of an exemplary method 500 for characterizing movement kinematics of exercises using a kinematic characteristic statistical model, according to some embodiments. In some embodiments, method 500 may be implemented in the kinematic characteristic statistical model, e.g., 124 (FIG. 1B), 200 (FIG. 2A), 240 (FIG. 2B). And thus, the acts in method 500 in the kinematic characteristic statistical model may be similar to those described in the various embodiments in FIGS. 1B, 2A, and 2B. For example, method 500 may include receiving sensor data from one or more sensors wearable on one or more body portions of a subject and/or configured to be disposed on a portion (e.g., moving portion) of a piece of exercise equipment, at act 502; and using a kinematic characteristic statistical model to determine one or more kinematic characteristics from the movement of the subject based on the sensor data, at act 506. It is appreciated that act 506 may include one or more acts corresponding to various components in the kinematic characteristic statistical model as shown in FIG. 2A.

In some embodiments, method 500 may optionally include using exercise identification statistical model to determine characteristics of exercises (e.g., type of exercise, and/or start/stop points) in the sensor data, at act 504, where determining the kinematic characteristics may be additionally based on the characteristics of exercise. In a non-limiting example, as described in FIG. 2A, the segmentation output 214, including, e.g., type of exercise, may be used by the metric calculation component 208 (e.g., principal selector 208-1). In a non-limiting example, as described in FIG. 2B, the characteristics of exercises information from exercise identification statistical model 230 may be incorporated and propagated in the movement kinematic machine learning model 240, which may be implemented in the kinematic characteristic statistical model 240, as described in embodiments in FIG. 2A.

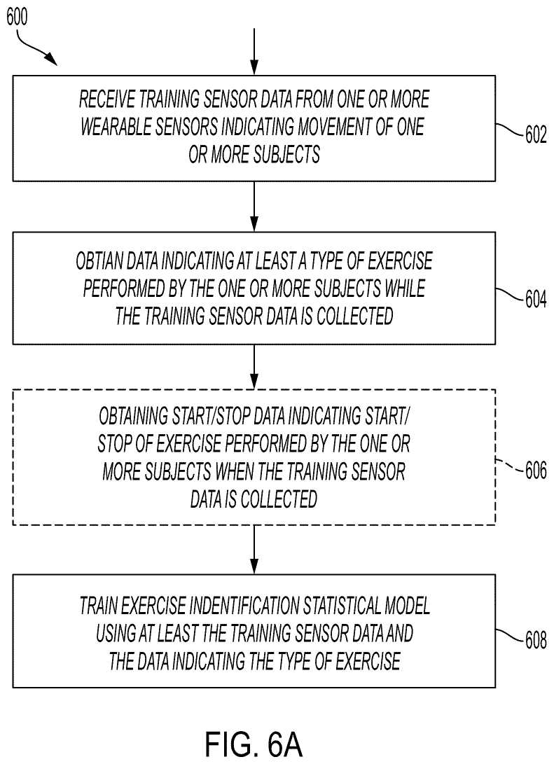

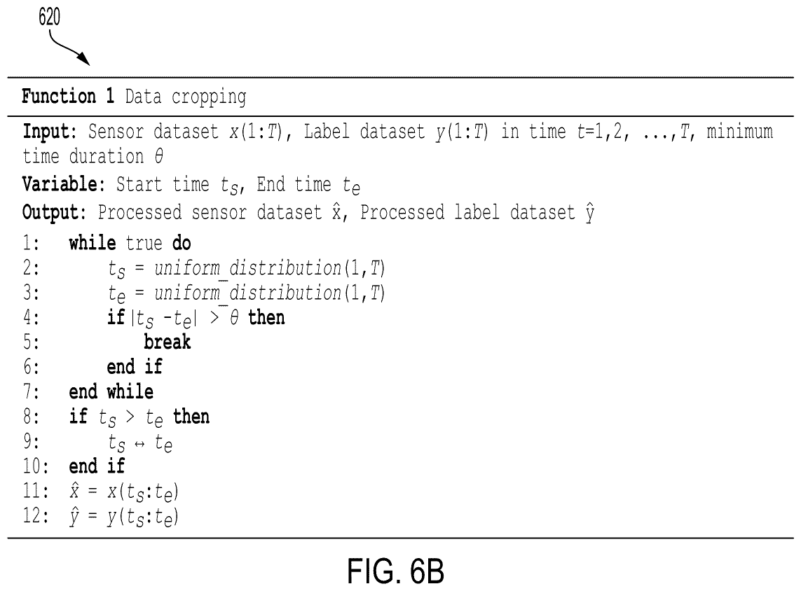

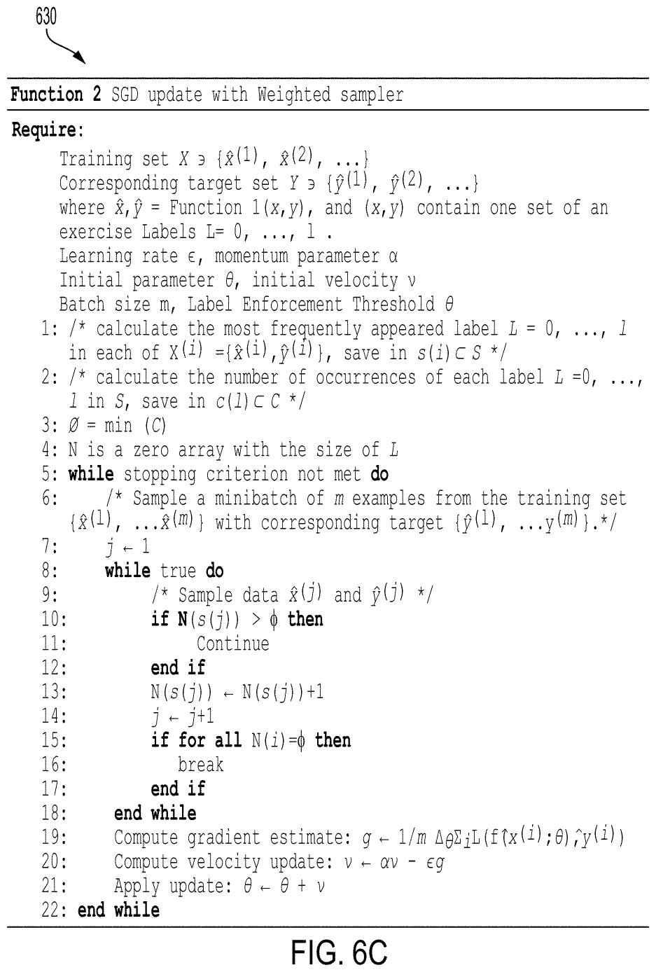

FIG. 6A is a flow diagram of an exemplary method 600 for training an exercise identification model, according to some embodiments. In some embodiments, method 600 may be implemented to train the exercise identification model, such as 102 (FIG. 1A), 122 (FIG. 1B), 230 (FIG. 2B). Thus, the training method may be similar to those described in embodiments in FIGS. 1A, 1B, and 2B. For example, method 600 may include receiving training sensor data collected at one or more sensors, at act 602, where the training sensor data indicating movement of one or more body portions of one or more subjects while performing one or more exercises. The training method 600 may further include obtaining data indicating at least a type of exercise performed by the one or more subjects while the training sensor data is collected, at act 604. Method 600 may further include training the exercise identification statistical model by using the received training sensor data and the data indicating the at least a type of exercise associated with the training sensor data, at act 608, where the trained exercise identification statistical model is configured to determine at least a type of exercise from a movement of a subject.

With further reference to FIG. 6A, in some embodiments, training method 600 may optionally include obtaining start/stop data indicating start/stop of exercise performed by the one or more subjects when the training sensor data is collected, at act 606. Thus, the exercise identification model may also be trained to further detect start point(s) and/or stop point(s) during the movement of the user.

In some embodiments, obtaining data indicating a type of exercise (at act 604) and obtaining data indicating start/stop of exercise (at act 608) may include obtaining ground truth data for the type of exercises, and optionally, the start/stop of exercises, when the training sensor data is collected. The ground truth data for the type of exercise may be provided during the collection of the training sensor data. For example, when an exercise is being performed by a user during collection of training sensor data, the type of exercise is known, and thus, can be pre-entered, or manually entered to be associated with the training sensor data. In some embodiments, the ground truth start/stop data may include one or more start points and one or more stop points. The one or more start points in the ground truth data may each indicate a start of an exercise when the training sensor data is collected, whereas the one or more stop points in the ground truth data may each indicate a stop of an exercise when the training sensor data is collected. The ground truth start/stop data may be provided by timing a start and/or stop of an exercise (e.g., an operator manually timing the start and/or stop) while the exercise is being performed by a user for collecting the training data.

FIGS. 6B-6C are flow diagrams of exemplary methods 620, 630 which can be used in a method 600 for training an exercise identification model, according to some embodiments. In some embodiment, method 620 may include a data cropping method to crop the data in the training sensor data. In the example method shown in FIG. 6B, sensor data set with a time duration of shorter than a threshold may be cropped. In some embodiments, with reference to FIG. 2B, the exercise identification model 230 may include stacked multiple 1-dimensional dilated convolutions with the window length from 1, 2, 4, . . . to 2ⁿ, where n is a user-defined parameter. For example, n may be 8, 9, 10, 12, or any other suitable number. The final layer of dilated convolution (e.g., 232-N) may be processed to have probability distribution over exercise types C. To train this model, Stochastic Gradient Descent (SGD) may be used to minimize an objective function, where the objective function may calculate class errors between the ground truth exercise types and predicted exercise types from the exercise identification statistical model. Method 630 includes a SGD update with weighted sampler that may be used in training the exercise identification model.

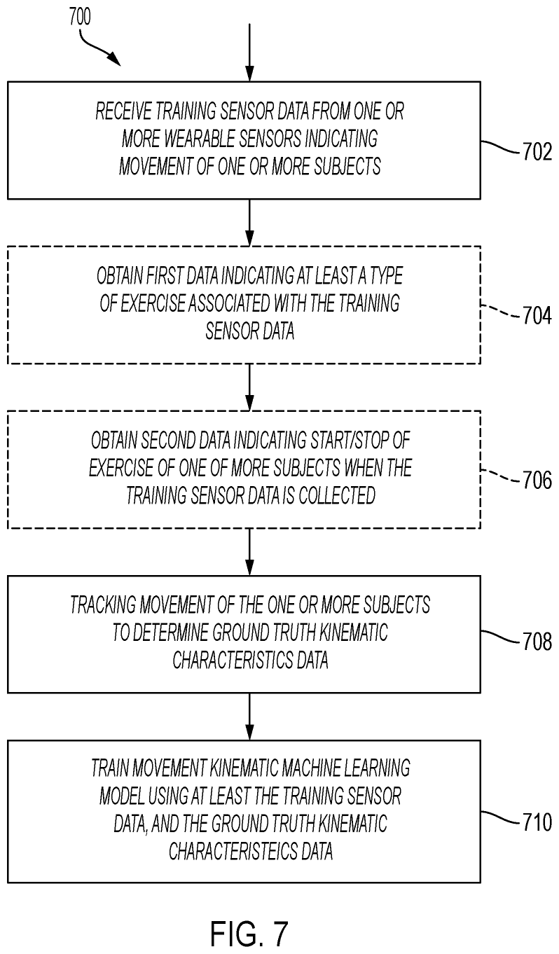

FIG. 7 is a flow diagram of an exemplary method 700 for training a movement kinematic machine learning model (e.g., 240 in FIG. 2B) that can be part of a kinematic characteristic statistical model, according to some embodiments. In some embodiments, method 700 may be implemented to train the movement kinematic machine learning model (e.g., 240 in FIG. 2B) that may be implemented in a kinematic characteristic statistical model (e.g., 124 (FIG. 1B), 200 (FIG. 2A). Thus, the training method may be similar to that described in embodiments in FIGS. 1B, 2A, and 2B. In some embodiments, method 700 may include receiving training sensor data collected at one or more, at act 702; tracking the movement of the one or more subjects while the training sensor data is collected to determine ground truth kinematic characteristics data, at act 708; and training the movement kinematic machine learning model by using the received training sensor data, and the ground truth kinematic characteristics data, at act 710. In some embodiments, the movement kinematic machine learning model may include a CNN, such as a dilated CNN, a stacked dilated CNN, as described above.

In some embodiments, tracking the movement of the subject(s) at act 708 may include a marker-based approach, which uses a motion capture system to track the positions of one or more markers placed on the body of the subject(s), and uses the tracked positions of the marker(s) to estimate the ground truth kinematic characteristics data (e.g., ground truth movement velocity and trajectory). Alternatively, and/or additionally, tracking the movement of the subjects may include using one or more cameras to track the positions and/or orientations of the one or more wearable sensors attached to the body of the subject(s). Subsequently, the training method may use the tracked positions and/or orientations of the sensor(s) to determine the ground truth kinematic characteristics data. The camera-based tracking is further described.

In some embodiments, tracking the movement of the one or more wearable sensors in act 708 using one or more cameras may include obtaining target points in global coordinates (x, y, z) in a camera frame. In some embodiments, the target points may include one or more points (for example, a symbol, e.g., a dot, placed around the sensor), which may be used to determine the position and orientation of the sensor. For example, three to four points may be placed around a sensor and used to calculate the xy vectors, and the z vector of the orientation of the sensor.

Act 708 may convert the target points obtained from cameras, in global coordinates to the sensor global coordinates (e.g., IMU global coordinates). The conversion of coordinates from camera coordinates to sensor coordinates may use various techniques. For example, an exemplary technique is described in Y. Jin et. al., “A Data-based Approach to Simultaneously Align Local and Global Frames between an Inertial Measurement Unit (IMU) and an Optical Motion Capture System,” 2022 9th IEEE RAS/EMBS International Conference for Biomedical Robotics and Biomechatronics (BioRob), Aug. 21-24, 2022, which is incorporated by reference herein.

In some embodiments, act 708 may further obtain global accelerations from the sensor global coordinates by differentiating the trajectory data from the target points. For example, the position data at time t and time t−1 may be differentiated once to determine the velocity. The velocity data may be further differentiated to determine the acceleration (in global coordinates), which may be used as input data to train the movement kinematic machine learning model.

Alternatively, and/or additionally, tracking the movement of the subjects at act 708 may include tracking the movement of each of the one or more subjects by receiving equipment movement data from a sensor installed on an exercise equipment on which the subject is exercising during the movement of the subject; and estimating the ground truth kinematic characteristics data based on the received data from the equipment movement data for the one or more subjects. For example, tracking the movement may include receiving sensor data from a linear variable differential transformer (LVDT) sensor installed on a bike, a treadmill, or other exercise equipment.

In some embodiments, in training the movement kinematic machine learning model 240, method 700 may additionally include obtaining first data indicating a type of exercise associated with the training sensor data at act 704. Method 700 may also include obtaining second data indicating start/stop of exercises of one or more subjects when the training sensor data is collected, at act 706. Subsequently, act 710 may be based additionally on the first and/or second data.

In some embodiments, obtaining the first data (act 704) and the second data (act 706) may be implemented using the exercise identification statistical model 230 (see FIG. 2B). It is appreciated that exercise identification statistical model 230 may be trained (as described in method 600 in FIG. 6A) separately from training method 700. In doing so, the parameters of the exercise identification statistical model are fixed while the movement kinematic machine learning model 240 is being trained.

Although it is shown that the output of the exercise identification statistical model may be used to reinforce learning of the movement kinematic machine learning model, it is appreciated that variations may be possible. For example, the output of the kinematic characteristic statistical model may be provided to the exercise identification statistical model to reinforce the learning of the exercise identification statistical model. In some variations, the regression output from the kinematic characteristic statistical model (see FIG. 2A) may be provided as input to the exercise identification statistical model 230 and used in the training of the exercise identification statistical model. In some variations, with reference to FIG. 2B, a final hidden layer of the movement kinematic machine learning model 240 may be provided as input to the exercise identification statistical model 230 and used in the training of the exercise identification statistical model. This will result in an improved accuracy of the exercise identification statistical model.

In some embodiments, training the movement kinematic machine learning model at act 710 may use the Root Mean Squared Error (RMSE) Loss function to minimize the Euclidean difference between ground truth measured by tracking of movement as described above and predicted velocity and trajectory in time domain. The learning rate can be defined empirically to minimize the loss.

As noted previously above, in some embodiments, a system may include a combined activity identification and kinematic characteristic statistical model, as described herein. The combined activity identification and kinematic characteristic statistical model may be implemented as separate activity identification and kinematic characteristic models which communicate information to each other during processing of recorded signals. However, in other embodiments, the combined activity identification and kinematic characteristic statistical model may be implemented as a single model. In some examples, the single combined model may include separate sections or layers for activity identification and for kinematic characteristics estimation.

In some examples, the separate models or sections of integrated models may be connected for the passing of information. For example, the output of a model or section of a model may be passed to the other model which again may be implemented as a separate model and/or as a section of the same model. In some examples, the identified activity may be passed to the kinematic characteristics section or model. In one such embodiment, the output of a layer or stack of layers may be passed from a section of a model to another section of the model. For example, the output of a stack of layers corresponding to an activity identification model or section may be passed to a stack of layers corresponding to a kinematics characteristic model. Additionally or alternatively, the output of a stack of layers corresponding to the kinematics characteristic model may be passed to a stack of layers corresponding to the activity identification model. Again, in some embodiments, these one or more layers corresponding to the different models may be implemented as a single integrated statistical model.

In some examples, a combined activity identification and kinematic characteristics model may be trained in one or more stages. In some examples, the model may be trained with a training dataset containing raw IMU measurements and ground truth activity identities and kinematic characteristics. However, instances in which preprocessing is applied to the sensor signals or other dataset are also contemplated. In some examples, the combined model may be trained in a single stage, with the activity identification and kinematic characteristics models or sections being trained together. In some examples, the combined model may be trained in multiple stages where the activity identification and kinematic characteristics models or sections are trained separately in different stages. In some examples, the combined model may be trained following separate training of the activity identification and kinematic characteristics models or sections of a single integrated model. In some examples, when different models or sections of models are being trained, the weights of the model not being trained may be locked, such that only the weights of the model, or portion of a model, being trained may change.

For the sake of clarity the below embodiments are described using exercise identification and characterization and specific exercise based examples are given. However, it should be understood that the disclosed methods and models, both below and elsewhere herein, may be used for any appropriate activity that may be identified and characterized by the disclosed methods and systems and that any reference to a model that is specific to an exercise may be generalized to other activities as well.

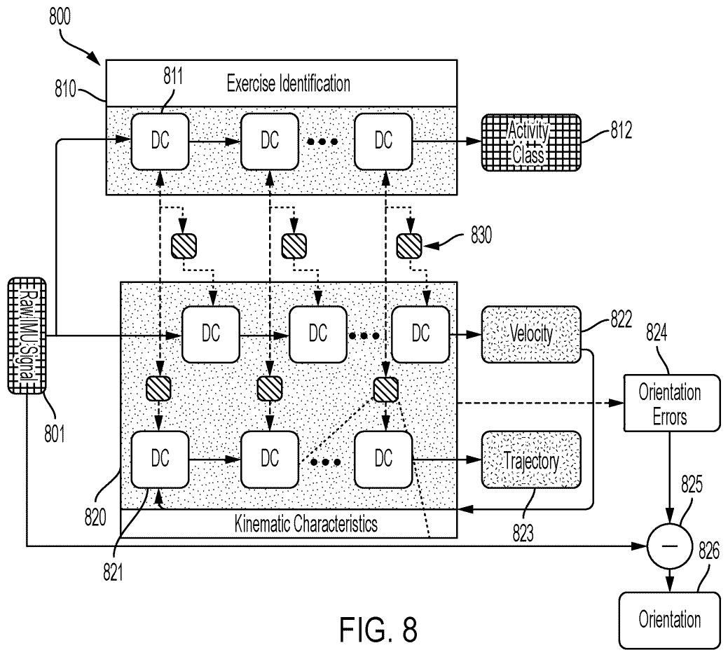

FIG. 8 is a diagram of an example combined model for kinematics estimation and activity classification, and specifically exercise identification, according to some embodiments of the technology described herein. Model 800 may analyze raw IMU signal 801, or other appropriate sensor signals, associated with the movement of one or more portions of a subject during exercise or other activity. The IMU signal may be recorded as described herein, for example as described with reference to FIG. 1C. In some examples, the IMU signals may be filtered, for example with high pass, low pass, or bandpass filters, among other filters. Model 800 includes exercise identification section 810 and kinematic characteristics section 820. The exercise identification section 810 and kinematic characteristics section 820 are connected with feature aggregation network 830 which may be configured to pass information between the different section in both directions as elaborated on further below. It should be appreciated that model 800 may be structured in other ways, such as with separate exercise identification and kinematic characteristics models or as a single model, as described herein.

The exercise identification section 810 and kinematic characteristics section 820 may both contain DCNNs for processing the IMU signals 801, however, it should be appreciated that other statistical models and/or model structures may be used, as described herein.

Exercise identification section 810 may be structured as multiple stacked DCNNs 811, or other types of models as described herein, which analyze raw IMU signal 801. The exercise identification section 810 may output an activity class 812, which may include an exercise type, or other activity depending on the embodiment, as described herein. In some examples, the activity class 812 may be passed to the kinematic characteristics section 820 or another section of the model 800.

Kinematic characteristics section 820 may be structured as multiple stacked DCNNs 821, though other models may again be used as described herein. As shown, a subset of the DCNNs 821 may be used to determine a velocity 822 from raw IMU signals 801. The determined velocity 822 may be used to determine a trajectory 823. For example, the output velocity, or other kinematic characteristic, may be input to one or more additional layers of the kinematic characteristics section 820 to determine another kinematic characteristic such as the noted trajectory 823. The kinematic characteristics section 820 may additionally determine orientation errors 824 from the raw IMU, or other appropriate sensor, signals 801. The orientation errors may be subtracted from the raw IMU signal 801 at 825 to determine orientation 826 of the IMU, and thus, the associated portion of the subjects body. While specific kinematic characteristics are shown being determined in FIG. 8, it should be appreciated that the kinematic characteristics section 820 may determine either different and/or additional kinematic characteristics as the disclosure is not so limited.

As noted above, a feature aggregation network 830 may be used to connect the exercise identification section 810 and kinematic characteristics section 820 of a combined statistical model in some embodiments. For example, the feature aggregation network may connect a DCNN 811 of the exercise identification section 810 to DCNNs 821 of the kinematic characteristics section 820. As shown, the feature aggregation network connects each DCNN 811 of the exercise identification section to two DCNNs of the kinematic characteristics section, however it should be appreciated that different connections are possible. For example, a DCNN may be connected to one DCNN of the other model or section, greater than one DCNN of the other model or section, greater than two DCNNs of the other model or section, or no DCNNs of the other model or section.

The feature aggregation network 830 passes data from the exercise identification section 810 to the kinematic characteristics section 820, and from the kinematic characteristics section 820 to the exercise identification section 810. For instance, in some embodiments, the feature aggregation network may pass the output of the final layer of a DCNN 811 of the exercise identification section 810 to the DCNNs 821 of the kinematic characteristics section 820. The DCNNs 821 of the kinematic characteristics section 820 may use this data in the determining of the kinematic characteristics from the raw IMU signal 801 or other appropriate sensor signal. As also shown in the figure, the feature aggregation network 830 may pass the output of the final layers of two DCNNs 821 of the kinematic characteristics section 820 to a DCNN 811 of the exercise identification section 810. The DCNNs 811 of the exercise identification section 810 may use this data in the determining of the activity class 812. In some embodiments, a feature aggregation network 830 may perform processing on the data being passed between an exercise identification section and a kinematic characteristics section, as described herein.

Without wishing to be bound by theory, the use of a feature aggregation network 830 may improve the accuracy of the determined activity class and kinematic characteristics, as additional data may be used by the exercise identification and kinematic characteristics sections when determining the respective outputs. Additionally, the depicted use of sequential feedback between the sequentially arranged layers of the different sections of the model may help to further improve the accuracy of the determined activity and kinematic characteristic information in the depicted algorithm and/or in other implementations.

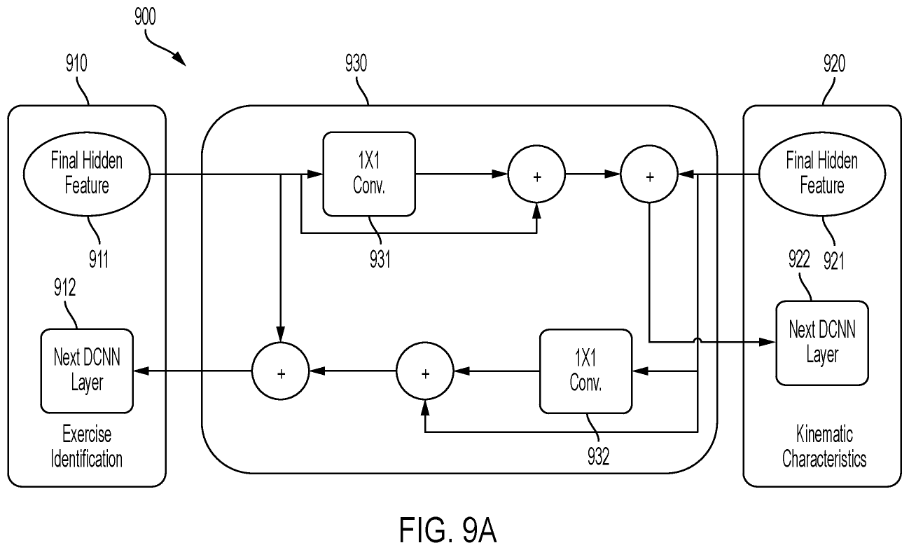

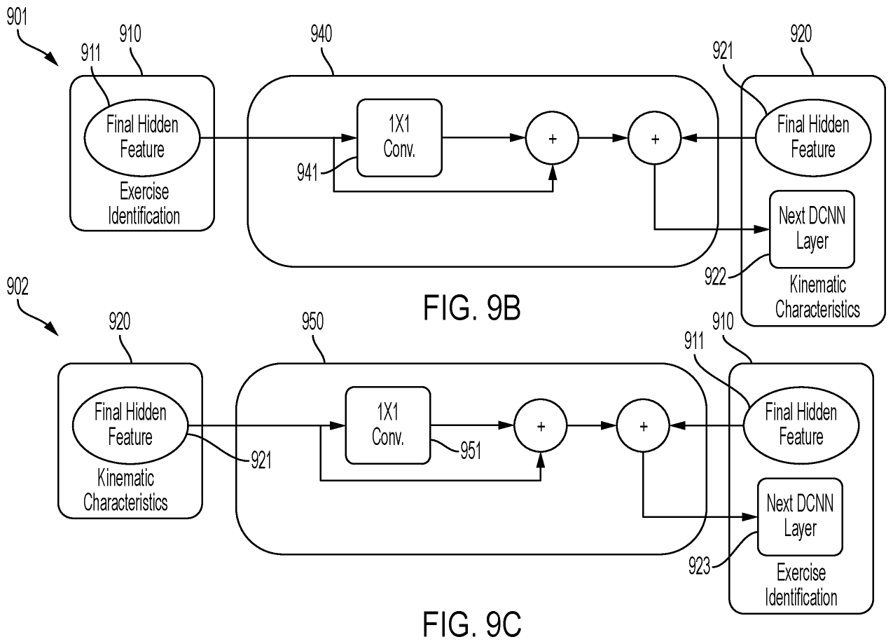

FIG. 9A is one example of an embodiment of a feature aggregation network for improving the accuracy of the exercise identification and kinematic characteristics estimation of a model, according to some embodiments of the technology described herein. The feature aggregation network 930 is configured to improve both the accuracy of the exercise identification and kinematic characteristics because it receives and passes data to both the exercise identification section 910 and the kinematic characteristics section 920 from the other corresponding section.

Similar to the embodiment of FIG. 8, a model 900 depicted in FIG. 9A includes exercise identification section 910, kinematic characteristics section 920 and feature aggregation network 930. As shown, the feature aggregation network is connecting a single layer of the exercise identification section 910 to a single layer of the kinematic characteristics section 920, however it should be appreciated that multiple layers may be connected in one section to multiple layers in another section by the feature aggregation network as described herein.

The model 900 may receive IMU data, or other appropriate sensor data as described herein. The IMU data may be collected, for example, as discussed with respect to FIG. 1C. The model 900 may be a combined exercise identification and kinematic characteristics model, as described herein. The model 900 may include multiple layers in the exercise identification section and the kinematic characteristic section which may analyze the IMU data and generate features. Each layer may generate one or more features from the IMU data, which are passed to subsequent layers for analysis. As shown, a final hidden feature 911 of a layer of the exercise identification section 910 is passed into feature aggregation network 930. Within the feature aggregation network 930, the final hidden feature may undergo processing, such as filtering or processing by one or more layers, as described herein. As shown, the final hidden feature may be passed to a 1×1 convolutional layer 931 followed by a rectified linear unit (ReLU). The processed final hidden feature may then be summed with the final hidden feature 921 of a layer of the kinematic characteristics section 920 and used as an input to the next DCNN layer 922 of the kinematic characteristics section 920.

The final hidden feature 921 of a layer of the kinematic characteristics section 920 may also be passed into the feature aggregation network 930, where it may be processed as described herein. As shown, the final hidden feature is passed to a 1×1 convolutional layer 932 followed by a ReLU. The processed final hidden feature is then summed with the final hidden feature 911 of a layer of the exercise identification section 910 and used as an input to the next DCNN layer 912 of the exercise identification section 910.

By passing final hidden features of layers of the exercise identification and kinematic characteristics sections through the feature aggregation network, the accuracy of the exercise identification and kinematic characteristics sections is improved. The accuracy is improved because the layers are receiving IMU data that has already been processed to identify features related to the exercise identity or kinematic characteristics of the exercise. Because the exercise identity and kinematic characteristics are closely related, for example certain exercises may be associated with specific trajectories, speeds, and angles, among other kinematic characteristics, the additional data passed by the feature aggregation network improves the prediction of both the exercise identification and kinematic characteristics sections.

FIG. 9B is another example of an embodiment of a feature aggregation network for improving the accuracy of estimation of kinematic characteristics, according to some aspects of the technology described herein. The model 901 includes exercise identification section 910, kinematic characteristics section 920 and feature aggregation network 940. The feature aggregation network 940 may function to improve the estimation of kinematic characteristics, as additional data, from the exercise identification section 910, is used in the determining of the kinematic characteristics by the kinematic characteristics section 920.

The model 901 may receive IMU data, or other appropriate sensor data, as described herein. The IMU data may be collected, for example, as discussed with respect to FIG. 1C. The model 901 may be a combined exercise identification and kinematic characteristics model, as described herein. The model 901 may include multiple layers in the exercise identification section and the kinematic characteristic section which may analyze the IMU data and generate features. Each layer may generate one or more features from the IMU data, which are passed to subsequent layers for analysis.

As shown, the final hidden feature 911 of a layer of the exercise identification section 910 may be passed to feature aggregation network 940. As shown, feature aggregation network 940 is connected to a single layer of the exercise identification section and the kinematic characteristics section, however it should be appreciated that the feature aggregation network 940 may include connections to multiple layers, as described herein. The final hidden feature is passed to a 1×1 convolutional layer 941 followed by a ReLU. The processed final hidden feature is then summed with the final hidden feature 921 of a layer of the kinematic characteristics section 920 and used as an input to the next DCNN layer 922 of the kinematic characteristics section 920.

FIG. 9C is yet another example of an embodiment of a feature aggregation network for improving the accuracy of exercise identification, according to some aspects of the technology described herein. The model 902 includes exercise identification section 910, kinematic characteristics section 920 and feature aggregation network 950. The feature aggregation network 950 may function to improve the estimation of exercise identification, as additional data, from the kinematic characteristics section 920, is used in the determining of the exercise identity by the exercise identification section 910.

The model 902 may receive IMU data, or other sensor data as described herein. The IMU data may be collected, for example, as discussed with respect to FIG. 1C. The model 902 may be a combined exercise identification and kinematic characteristics model, as described herein. The model 902 may include multiple layers in the exercise identification section and the kinematic characteristic section which may analyze the IMU data and generate features. Each layer may generate one or more features from the IMU data, which are passed to subsequent layers for analysis.

As shown, the final hidden feature 921 of a layer of the exercise identification section 920 is passed to feature aggregation network 950. As shown, feature aggregation 950 is connected to a single layer of the exercise identification section and the kinematic characteristics section, however it should be appreciated that the feature aggregation network 950 may include connections to multiple layers, as described herein. The final hidden feature is passed to a 1×1 convolutional layer 951 followed by a ReLU. The processed final hidden feature is then summed with the final hidden feature 911 of a layer of the exercise identification section 910 and used as an input to the next DCNN layer 912 of the exercise identification section 910.

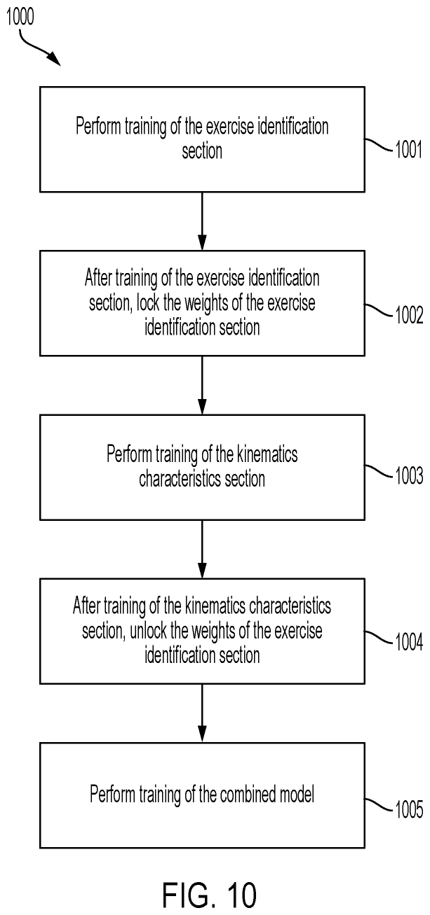

FIG. 10 is an example of one embodiment of a process for training a combined exercise identification and kinematic characteristics model, according to some aspects of the technology described herein. The process 1000 may be used to train a combined exercise identification and kinematic characteristics model, as described herein. The combined exercise identification and kinematic characteristics model may include a feature aggregation network, as described herein. The combined exercise identification and kinematic characteristics model may be structured according to any of the embodiments described herein, for example with separate models for exercise identification and kinematic characteristics, separate sections for exercise identification and kinematic characteristics, or as a combined model.

The process 1000 begins at step 1001 in which the exercise identification section is trained. The exercise identification section may be trained by using a training dataset containing IMU measurements, or other appropriate sensor data, and ground truth exercise identities, or other types of activities, and kinematic characteristics. In some examples, the training dataset may include raw or filtered IMU measurements. In some examples, the ground truth data from the dataset may be obtained from measurements by a motion capture camera system, manual identification, separate sensor datasets and associated traditional analysis, and/or using any other appropriate method for obtaining the desired ground truth data as the disclosure is not so limited.

In step 1001, the exercise identification section may be trained by analyzing the training dataset using the exercise identification section of the model, comparing the results of the analysis to the ground truth data and adjusting one or more weights of the model based on the comparison between the ground truth data and the prediction of the exercise identification section. It should be appreciated that similar training may be performed for exercise identification for a combined model having separate exercise identification and kinematic characteristics sections and for a combined model having a single model. In some examples, the weights may be adjusted such that a loss function is minimized, such as a cross entropy loss, a root mean square error, a hinge loss, a mean absolute error, a huber loss, a log-cosh loss, a quantile loss, or a categorical cross entropy loss, among other loss functions may be used. In some examples, the categorical cross entropy loss is used to minimize the classification error between the ground truth data and the prediction of the exercise identification section. The exercise identification section may be trained until the weights of the model converge, a predetermined number of training iterations have been performed, or the error between the prediction of the exercise identification section and the ground truth data is below a threshold level. For example, in some examples, the exercise identification section may be trained as described herein, for example, with respect to FIGS. 6A-6C.

The process may then proceed to step 1002, in which the weights of the exercise identification section are locked. When the weights are locked, their values may not be changed during subsequent training steps until they are unlocked. The process may then proceed to step 1003, in which the kinematic characteristics section of the model is trained. The kinematic characteristics section of the model may be trained by analyzing the training dataset using the kinematic characteristics section of the model, comparing the results of the analysis to the ground truth data and adjusting one or more weights of the kinematic characteristics section based on the comparison between the ground truth data and the prediction of the kinematic characteristics section. It should be appreciated that similar training may be performed for kinematic characteristics for a combined model having separate exercise identification and kinematic characteristics sections and for a combined model having a single model. In some examples, the weights may be adjusted such that a loss function is minimized, such as a cross entropy loss, a root mean square error, a hinge loss, a mean absolute error, a Huber loss, a log-cosh loss, a quantile loss, or a categorical cross entropy loss, among other loss functions may be used.

In some examples, different loss functions may be minimized based on the kinematic characteristic being predicted. In some examples, for every layer of DCNN, in experimentally implemented embodiments, a Mean Squared Error function was used to minimize the error between estimated and ground truth velocities and trajectories, according to the below equation:

$$L = \frac{1}{N} ( {\sum }_{i = 1}^{N} {( V - \hat{V} )}^{2} + {\sum }_{i = 1}^{N} {( Φ - \hat{Φ} )}^{2} )$$

where V and Φ is ground truth velocity and trajectory that can be obtained from motion capture cameras, and \hat{V} and \hat{Φ} is predicted velocity and trajectory based on the IMU data of the training dataset. In some examples, for joint angle estimation, the following loss function may be used to minimize angular error, based on the quaternion inner product,

$$L = \frac{1}{N} {\sum }_{i = 1}^{N} arc cos ( ( \vert {q}_{gt} \cdot {q}_{p r e d} \vert )$$

where N is the number of samples, q_{gt} is the ground truth quaternion obtained from the Mocap and q_{pred} is the model-predicted quaternion after normalization. The kinematic characteristics section may be trained to identify one or more kinematic characteristics as described herein.

While specific functions are shown above, it should be understood that other appropriate functions may be used during the training process. In either case, the kinematic characteristics section may be trained until the weights of the model converge, a predetermined number of training iterations have been performed, or the error between the predictions of the kinematic characteristics and the ground truth data is below a threshold level.

The process may then proceed to step 1004, in which the weights of the exercise identification section are unlocked, such that they may be changed in subsequent training steps. The process may then proceed to step 1005, in which training of the combined model is performed. The combined model may be trained by analyzing the IMU data, or other appropriate sensor data, of the training dataset with the exercise identification section and the kinematic characteristics section, comparing the predictions of the exercise and kinematic characteristics to the ground truth data and adjusting one or more weights of the model based on the comparison. In some examples, the weights may be adjusted such that a loss function is minimized, such as a cross entropy loss, a root mean square error, a hinge loss, a mean absolute error, a Huber loss, a log-cosh loss, a quantile loss, or a categorical cross entropy loss, among other loss functions may be used. The combined model may be trained until the weights of the model converge, a predetermined number of training iterations have been performed, or the error between the predictions of the combined model and the ground truth data is below a threshold level.

In some examples, the exercise identification section may be trained after the kinematic characteristics section. In some examples, the exercise identification section may be trained for 500 epochs. In some examples the exercise identification section may be trained for greater or fewer than 500 epochs, for example 10 epochs, 50 epochs, 100 epochs, 250 epochs, 700 epochs, 1000 epochs, 5000 epochs, 10-5000 epochs or greater than 5000 epochs. In some examples, the kinematic characteristics section may be trained for at least 1000 epochs. In some kinematic characteristics section may be trained for greater or fewer than 1000 epochs, for example, 10 epochs, 50 epochs, 100 epochs, 250 epochs, 500 epochs, 700 epochs, 5000 epochs, 10-5000 epochs or greater than 5000 epochs. In some examples, the exercise identification section and the kinematic characteristics section have a 4 stage architecture, with each stage having a number of layers, for example eleven, dilated convolutional layers with respective dilation lengths of 1, 2, 4, 8, 16, 32, 64, 128, 256, 512, or other appropriate value with a kernel size of 3 or other appropriate size. In some examples, greater or fewer numbers of layers may be used such as 1 layer, 5 layers, 10 layers, 20 layers, 25 layers, 50 layers, between 1 and 50 layers, or greater than 50 layers. In some examples, the hidden dimension is 64 for all layers including AAN. In some examples the Adam optimizer may be used in training with a learning rate of 10⁻⁴ and weight decay of 10⁻⁷. Of course, while a particular layer architecture is described above, it should be understood that different numbers of layers with different configurations and/or different types of models may be used as the disclosure is not so limited.

### Example: Strength Training Analysis

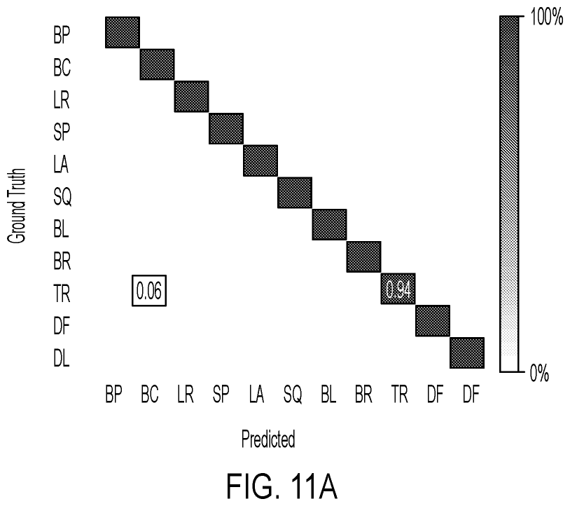

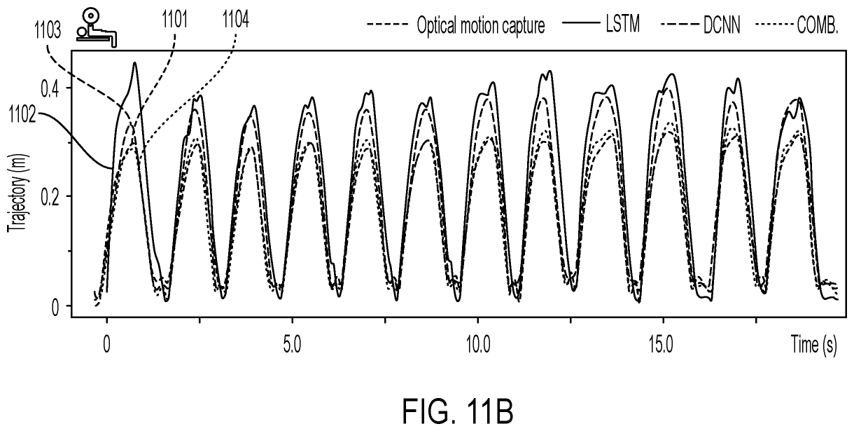

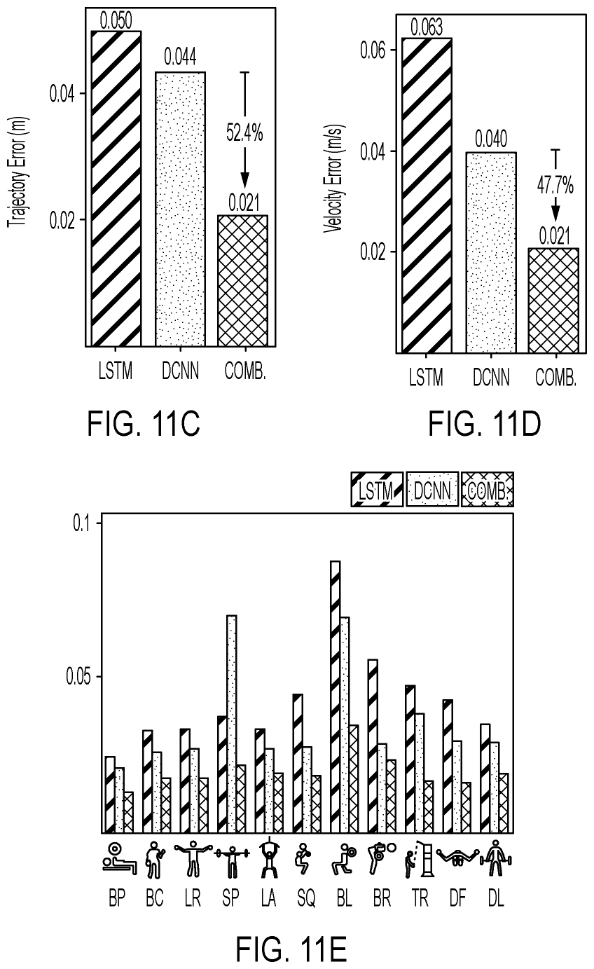

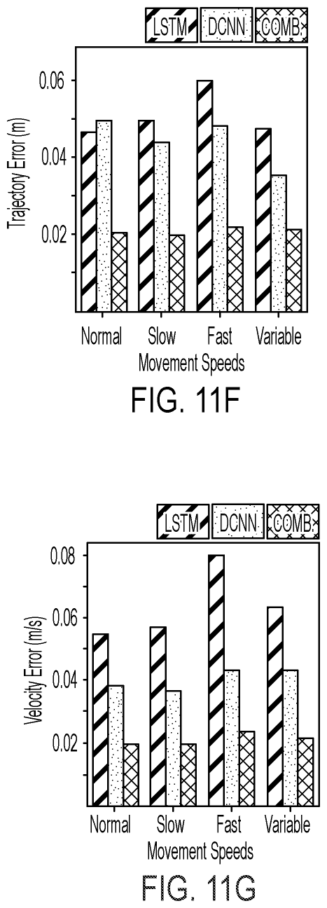

FIGS. 11A-11G depict data collected during an experimental process for exercise identification and kinematic characteristic estimation, using the above-described system and methods, according to one embodiment. The data of FIGS. 11A-11G was generated by and/or analyzed by a combined exercise identification and kinematic characteristics model similar to those described additional. Additional models were trained for the classification of activities and the analysis of kinematics, including a DCNN-based model which does not include a feature aggregation network or an exercise identification section, a LSTM model, and the Xsens joint angle fusion algorithm.

In the experimental process, data were collected on fifteen healthy participants (3 females; 28.1±5.6) with two IMUs, one placed on their chest and one on left wrist. The participants were asked to perform four sets of 11 different strength training exercises with 12 repetitions per set. The four sets were performed at four different self-selected movement speeds, normal, slow, fast, and variable. The beginning and the end of each exercise were segmented by pressing a button, which was time-synchronized with the IMU and motion capture cameras. Each IMU (Bosch BNO0030, Bosch, Germany) was mounted on a custom 3D printed case with four Motion capture markers on each of the corners. The IMUs were connected to a system including an appropriate processor, specifically a Beaglebone Black (Texas Instrument, USA) was used, to collect 3-axis acceleration, a-axis gyroscope, and 4-axis quaternion at 100 Hz. The quaternion values were obtained from the internal Kalman filter of the IMUs. IMU and Optical Motion Capture (OMC) data were time-synchronized using a 5V analog trigger signal. This also provides times for the start and end of an exercise for inclusion in the dataset which were used for classification.

The trajectory data from motion capture cameras was used as the ground truth in the training of the combined exercise identification and kinematics estimation model. To ensure the correct alignment between the IMU and the mocap measurements, the local and global frames were aligned between the two systems using a frame alignment method. The IMU global frame was defined using gravity and the magnetic north. The Mocap local frame (OL) was defined by the markers on the IMU case, which was assumed as the IMU global frame. The Mocap global frame (OG) was defined using a calibration tool. A calibration tool, is commonly used in motion capture systems and usually placed flat on the ground in alignment with floor tiles or walls for cameras to register it as the Mocap global coordinate system.

Before collecting data, a 1-minute calibration trial was performed, where participants performed random motions, to find the rotation matrix between OL and OG. The Mocap data were used as ground truth of our machine learning models. The experimental data were then collected as described above.

Data from 11 participants was used for training of the classification model. The exercise identification section of the model was trained for 500 epochs. The weights of the exercise identification section were then fixed and the kinematic characteristic section was trained for 1000 epochs. The exercise identification section and the kinematic characteristics section had four stage architecture with eleven dilated convolutional layers in each stage with a kernel size of three. The hidden dimension was set to 64 for all layers and the Adam optimizer was used with a learning rate of 10⁻⁴ and a weight decay of 10⁻⁷. Data from three randomly selected participants were used as the test dataset to evaluate the model; the data from those participants were not used for training or validation purposes.

FIG. 11A depicts a confusion matrix for the identification of strength training exercises during an experimental process, according to one embodiment. The model achieved an overall classification accuracy of 99.6%, when compared to the ground truth data. The model demonstrated 100% accuracy across all the exercise labels except for triceps extension. Triceps extensions were confused with Biceps curls approximately 6% of the time. The high accuracy of the exercise identification indicates the ability of the combined model to accurately identify exercises with a low number of sensors.

FIG. 11B depicts an example of a trajectory for a biceps curl exercise during an experimental process, according to one embodiment. FIG. 11B includes trajectory 1101 determined from optical motion capture, trajectory 1102 determined from an LSTM model, trajectory 1103 determined from a DCNN-based model without a feature aggregation network or an exercise identification section, and trajectory 1104 determined from a combined exercise identification and kinematics estimation model as described herein. The trajectory may be analysed to determine errors between the ground truth measurements and the estimated trajectory as determined by the ML models.

FIG. 11C depicts a graph of trajectory error for different models determined from an experimental process, according to one embodiment. The combined exercise identification and kinematics characteristic model achieved an error of 0.021 m as compared to DCNN and LSTM with 0.044 m and 0.05 m, respectively. The error of AIL-KE was 52% and 58% lower than the errors of DCNN and LSTM respectively. The errors of FIG. 11C were obtained by averaging the estimation errors from both chest and wrist IMUs. The reduced errors of the combined model when compared to the DCNN and LSTM indicates the ability of the system to determine kinematic characteristics with improved accuracy and fewer sensors than conventional techniques.

FIG. 11D depicts a graph of velocity error for different models, determined from an experimental process, according to one embodiment. The combined exercise identification and kinematic characteristics model achieved a velocity error of 0.020 m/s as compared to DCNN with 0.040 m/s and LSTM with 0.063 m/s. The error of the combined exercise identification and kinematic characteristics model was 48% and 67% lower than the errors of DCNN and LSTM respectively. The errors of FIG. 11D were obtained by averaging the estimation errors from both chest and wrist IMUs. The reduced errors of the combined model when compared to the DCNN and LSTM indicates the ability of the system to determine kinematic characteristics with improved accuracy and fewer sensors than conventional techniques.

FIG. 11E depicts a graph of trajectory errors across different exercises for different models, determined from an experimental process, according to one embodiment. The exercises include bench press, bicep curl, lateral raise, standing press, “lat” pulldown, squat, barbell lunge, bent-over row, triceps pushdown, dumbell fly, and deadlift. The errors for the LTSM model (left most bar), DCNN model (center bar) and combined exercise identification and kinematic characteristics model (rightmost bar) are shown for each exercise. The combined model had the lowest error for all exercises. Within these exercises, the combined model had the largest error for the barbell lunge exercise (0.034 m) across all the approaches. Still, this error was lower than the errors of 0.069 m for DCNN and 0.088 m for LSTM. For all the other strength training exercises, the combined model had errors lower than 0.03 m. The reduced errors of the combined model when compared to the DCNN and LSTM indicates the ability of the system to determine kinematic characteristics with improved accuracy and fewer sensors.

In strength training, movement velocity can be significantly different depending on an individuals' workout strategy and the level of fatigue. Therefore, it may be desirable to validate the performance of the models on different movement speeds.

FIG. 11F depicts a graph of trajectory error for different models at different movement speeds, determined from an experimental process, according to one embodiment. The trajectory error is shown for the LSTM (leftmost bar), DCNN (middle bar) and combined exercise identification and kinematic characteristic model (rightmost bar), for each of a normal, slow fast and variable movement speed. The combined model showed the lowest error across all movement speeds. For the combined model, trajectory errors for fast speed were worse when compared to errors for the other movement speeds, displaying the RMSE of 0.022 m. Still, these errors for fast speed were only 0.002 m than the average errors of combined model across all the speeds. The trajectory errors of the combined model for fast speed were 55.1% and 63.8% lower than the errors of DCNN and LSTM for fast speed respectively. The reduced errors of the combined model when compared to the DCNN and LSTM indicates the ability of the system to determine kinematic characteristics with improved accuracy and fewer sensors across different speeds, compared to conventional techniques.

FIG. 11G depicts a graph of velocity error for different models at different movement speeds, determined from an experimental process, according to one embodiment. The velocity error is shown for the LSTM (leftmost bar), DCNN (middle bar) and combined exercise identification and kinematic characteristic model (rightmost bar), for each of a normal, slow fast and variable movement speed. The combined model showed the lowest error across all movement speeds. For the combined model, velocity errors for fast speed were worse when compared to errors for the other movement speeds, displaying the RMSE of 0.024 m/s. Still, these errors for fast speed were only 0.003 m/s higher than the average errors of the combined model across all the speeds. For velocity estimation, the combined model had 45% and 70.1% lower errors compared with DCNN and LSTM respectively.

Overall, the combined exercise identification and kinematic characteristics model outperformed the other two models at all speeds. The combined model had a trajectory error standard deviation of 0.0007 (m) across all movement speeds. This value was one-seventh as low as that of DCNN and LSTM, indicating that combined model had lower variability in performance across different speeds. To assess the sensitivity of the combined model to individual variability, its performance on participants that were not in the training data was tested. The standard deviation values of the errors of the generalized model across participants was as low as 6.6×10−5 meters for three participants for the wrist IMU, which indicates the model can be adapted without user specific calibration for accurate exercise identification and kinematic characteristics estimation. The Normalized Root-Mean-Square Deviation (NRMSD), which evaluates the dispersion of data across participants, was lower than 4% for both trajectory and angle estimation by the combined exercise identification and kinematic characteristics model. An NRMSD value closer to 0 indicates that the errors across participants are similar. The NRMSDs for the combined model estimates were less than half of those from DCNN, which support the use of the model across individuals without additional person-specific calibration.

In addition to the above, the RMSE of the combined exercise identification and kinematic characteristics model for joint angle estimation at the end of 10 minutes (the last minute of the trial) was 5.33 degrees, over 2 degrees lower than the other techniques. Other techniques have obtained root mean squared differences higher than 5 degrees between the first and the 10th minutes for all joint angles over a ten-minute period. The approach described herein displayed root mean squared differences less than 1 degrees between the first and the last minutes. These results suggest that the technique described herein is robust to drift over the span of 10 minutes.

### Example: Orientation Estimation During Work Tasks

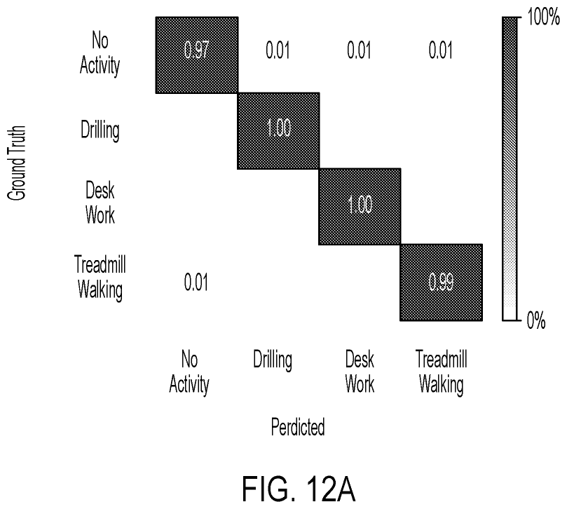

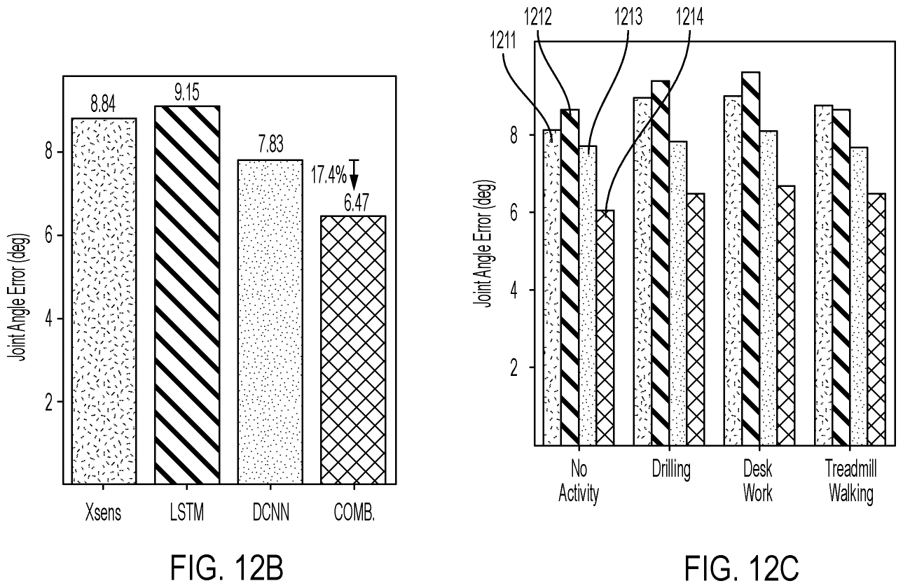

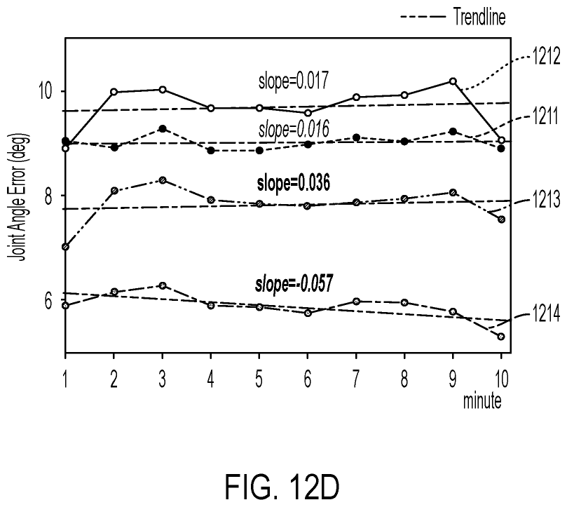

FIGS. 12A-12D depict data collected during an experimental process for activity identification and kinematic characteristic estimation, using the above-described system and methods, according to one embodiment. The data of FIGS. 11A-11G was generated by and/or analyzed by a combined exercise identification and kinematic characteristics model, as described herein. Additional models were trained for the classification of activities and the analysis of kinematics, including a DCNN-based model which does not include a feature aggregation network or an exercise identification section, a LSTM model, and the Xsens joint angle fusion algorithm.

In the experimental process, six participants (all male; 30±5.5) wore 3 IMUs, one on their chest and another two on their right and left upper arms. They were asked to perform functional activities including overhead drilling, desk work, and treadmill walking, simulating a typical industrial assembly workflow. Each task was completed in three-minute intervals, totaling more than 10 minutes for each trial with breaks and transitions included. In total, each participant performed five trials of activities with approximately one hour of data collected. We used data from two IMUs (chest-right arm or chest-left arm) as inputs to AIL-KE. Each participant performed 5 sets of overhead drilling, desk work (such as typing and note-taking), and treadmill walking. Each task was 3 minutes. “No activity” was provided as an additional label to indicate any activities performed transitioning among the three tasks. Xsens MTI-3 IMUs were used and collected data at 50 Hz. Each IMU was mounted on a custom 3D printed case with 4 Motion capture markers on each of the corners. IMU and Optical Motion Capture (OMC) data were time-synchronized using a 5V analog trigger signal. This also provides times for the start and end of a functional activity, which were used for classification. The IMU global frame was defined using gravity and the magnetic north. The Mocap local frame (OL) was defined by the markers on the IMU case, which was assumed as IMU global frame. The Mocap global frame (OG) was defined using a calibration tool. A calibration tool, like an L-frame, is commonly used in motion capture systems and usually placed flat on the ground in alignment with floor tiles or walls for cameras to register it as the Mocap global coordinate system.

Before collecting data, a 1-minute calibration trial was performed, where participants performed random motions, to find the rotation matrix between OL and OG. The Mocap data were used as ground truth of our machine learning models. The data was then collected as described above.

The orientation of arm relative to torso was calculated from the recorded data using the following equation: R^{torso}ₐᵣₘ=(R^{OMC}ₜₒᵣₛₒ)^{T}R^{OMC}ₐᵣₘ, where R^{OMC}ₜₒᵣₛₒ represents the rotation matrix of the torso IMU's local frame expressed in mocap global frame and R^{OMC}ₐᵣₘ represents the rotation matrix of the arm IMU's local frame expressed in mocap global frame Joint angle data obtained and processed from motion capture cameras was used as the ground truth of the combined exercise identification and kinematic characteristics model.

Leave-One-Out Cross Validation (LOOCV) was performed to assess the generalization ability across participants. Data from each participant was used as a test dataset to evaluate the model's performance. The average errors across participants were reported for each machine learning model, including LSTM and DCNN. Data from four participants was used for training and data from the last person was used for validation. The exercise identification section of the model was trained for 500 epochs. The weights of the exercise identification section were then fixed and the kinematic characteristic section was trained for 1000 epochs. The exercise identification section and the kinematic characteristics section had four stage architecture with eleven dilated convolutional layers in each stage with a kernel size of three. The hidden dimension was set to 64 for all layers and the Adam optimizer was used with a learning rate of 1e-4 and a weight decay of 1e-7.

FIG. 12A depicts a confusion matrix for the identification of activities during an experimental process, according to one embodiment. The combined exercise identification and kinematic characteristics model achieved an overall classification accuracy of 99.8%. The high accuracy of the exercise identification indicates the ability of the combined model to accurately identify activities with a low number of sensors.

FIG. 12B depicts a graph of results for joint angle error for different models determined during an experimental process, according to one embodiment. The estimation results for 3D joint angles of the right and left upper arms were evaluated. The combined exercise identification and kinematics estimation model was compared against three methods: LSTM, DCNN, and XSens. In XSens, the angular difference between the chest and left/right upper arms was calculated using the angles directly outputted from the XSens′ proprietary filters. Overall, the combined exercise identification and kinematics estimation model achieved an average estimation error of 6.5 degrees, as compared to DCNN with 7.83 degrees, LSTM with 9.15 degrees, and XSens with 8.84 degrees. The combined model generated best performance with angular errors being 17.4%, 29.3%, and 26.8% lower than LSTM, DCNN, and XSens respectively, highlighting the effectiveness of learning-based approaches in reducing angle estimation errors. The reduced errors of the combined model when compared to the XSens, DCNN and LSTM indicates the ability of the system to determine kinematic characteristics with improved accuracy and fewer sensors than conventional techniques.

FIG. 12C depicts a graph of angle error for different models across different activities, determined during an experimental process, according to one embodiment. The graph includes bars 1201 corresponding to XSens errors, 1202 corresponding to LSTM errors, 1203 corresponding to DCNN errors and 1204 corresponding to combined exercise identification and kinematic characteristics error. The bars 1201, 1202, 1203, and 1204 are shown for no activity, drilling, desk work and treadmill walking. The combined model had the lowest error across all activities and had a standard deviation of 0.25 degree across all activities. The reduced errors of the combined model when compared to the XSens, DCNN and LSTM indicates the ability of the system to determine kinematic characteristics with improved accuracy and fewer sensors than conventional techniques. Overall, the RMSE of the combined exercise identification and kinematic characteristics model at the shoulder joint was less than 6 degrees. Given that the range of joint angles for typical hand/tool positionings during overhead work is reported to be 70 degrees, this performance corresponds to less than 10% error across the range of motion.

FIG. 12D depicts a graph of joint angle error for different models across different activities over time, determined during an experimental process, according to one embodiment. The graph includes lines 1211 corresponding to XSens errors, 1212 corresponding to LSTM errors, 1213 corresponding to DCNN errors and 1214 corresponding to combined exercise identification and kinematic characteristics error. The errors are shown over a 10 minute period. The change in error over the 10 minute period is indicative of long term drift in measurements. The combined exercise characteristic and kinematic characteristics model demonstrated negative trendline slope from the first minute to the last minute with the lowest joint angle errors across all minutes compared to the other models. The reduced error over tome of the combined model when compared to the XSens, DCNN and LSTM indicates the ability of the system to determine kinematic characteristics with improved accuracy and fewer sensors and reduce the impact of long term drift on measurement and estimation accuracies. The performance of the combined exercise identification and kinematic characteristic model was additionally evaluated for longer than 10-minutes and found that estimation accuracy during the last minute was at least 20% better than with the other approaches investigated.

In view of the above results, the combined exercise identification and kinematic characteristics model provides accurate information of shoulder elevation angles of an individual against long-term drift, which is desirable for ergonomics applications, such as risk assessment and injury prevention.

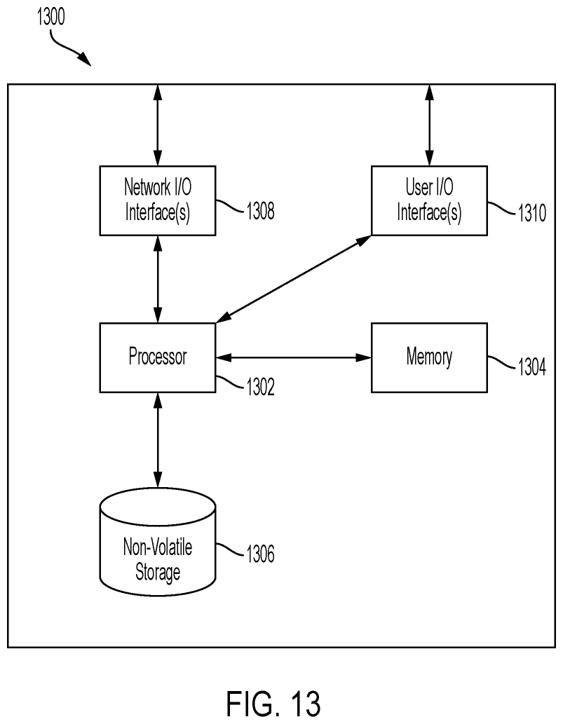

FIG. 13 shows an illustrative implementation of a computer system that may be used to perform any of the aspects of the techniques and embodiments disclosed herein, according to some embodiments. For example, the computer system 1300 may be configured to implement any of the exercise identification statistical model (102 in FIG. 1A, 122 in FIG. 1B, 230 in FIG. 2B), the kinematic characteristic statistical model (e.g., 124 in FIG. 1B, 200 in FIG. 2A), the movement kinematic machine learning model (e.g., 240 in FIG. 2B), the combined exercise identification and kinematic characteristics models (e.g. 800 in FIG. 8, 900 in FIG. 9A, 901 in FIG. 9B, or 902 in FIG. 9C) or any of the methods 400 (FIG. 4), 500 (FIG. 5), 600, 620, 630 (FIGS. 6A-6C), 700 (FIG. 7), or method 1000 (FIG. 10). The computer system 1300 may include one or more processors 1302 and one or more non-transitory computer-readable storage media (e.g., memory 1304 and one or more non-volatile storage media 1306) and a display 1310. The one or more processors 1302 may control writing data to and reading data from the memory 1304 and the non-volatile storage device 1306 in any suitable manner, as the aspects of the invention described herein are not limited in this respect. To perform functionality and/or techniques described herein, the processor(s) 1302 may execute one or more instructions stored in one or more computer-readable storage media (e.g., the memory 1304, storage media, etc.), which may serve as non-transitory computer-readable storage media storing instructions for execution by the processor 1302.

In connection with techniques described herein, code used to, for example, train and/or run any one or more of the models and/or methods described in the present disclosure may be stored on one or more computer-readable storage media of computer system 1300. Processor(s) 1302 may execute any such code to provide any techniques for detecting anomalies as described herein. Any other software, programs or instructions described herein may also be stored and executed by computer system 1300. It will be appreciated that computer code may be applied to any aspects of methods and techniques described herein. For example, computer code may be applied to interact with an operating system to detect anomalies through conventional operating system processes.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a cloud or other framework via a network interface 1308.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A system for activity identification and characterizing movement kinematics, the system comprising:
at least one processor configured to:
receive sensor data from two or more sensors indicating movement of two or more body portions of a subject during movement of the subject;
use a statistical model to determine a type of activity during the movement of the subject, based in part on the sensor data; and
use the statistical model to determine one or more kinematic characteristics during the movement of the subject, based in part on the sensor data and the type of activity.

2. The system of claim 1, wherein the statistical model simultaneously determines the type of activity and the one or more kinematic characteristics.

3. The system of claim 1, wherein the activity is an exercise.

4. The system of claim 1, wherein the two or more sensors are attachable to one or more pieces of exercise equipment.

5. The system of claim 1, wherein the two or more sensors are wearable on one or more body portions of the subject.

6. The system of claim 1, wherein:
the sensor data comprises at least first data and second data respectively received from the at least two sensors, wherein the first data and the second data are time-synchronized.

7. The system of claim 6, wherein the at least two sensors include a first sensor attached to a proximal portion of the subject and a second sensor attached to a distal portion of the subject.

8. The system of claim 7, wherein:
the proximal portion of the subject comprises a torso, a thigh, or a proximal portion of a limb of the subject; and
the distal portion of the subject comprises an end of a limb of the subject.

9. The system of claim 7, wherein the sensor data comprises at least a difference between a first reading from the first sensor and a second reading from the second sensor.

10. The system of claim 1, wherein the at least one processor is further configured to:
use the statistical model to additionally determine at least a start point and/or a stop point during the movement of the subject.

11. The system of claim 1, wherein the sensor data includes at least one or more of: acceleration, angular velocity, or magnetometer data.

12. The system of claim 1, wherein the statistical model comprises a dilated convolutional neural network.

13. The system of claim 1, wherein the statistical model, after determining the type of activity and the one or more kinematic characteristics, redetermines the type of activity based in part on the one or more kinematic characteristics.

14. The system of claim 1, wherein the statistical model determines the type of activity from a predetermined list of activities.

15. The system of claim 10, wherein the statistical model determines a start point and/or a stop point of an activity.

16. The system of claim 1, wherein the at least one processor is configured to determine the one or more kinematic characteristics by at least estimating movement velocity and trajectory from the sensor data.

17. The system of claim 16, wherein the at least one processor is configured to estimate the movement velocity and trajectory from the sensor data by:
estimating the movement velocity based on the sensor data;
filtering the estimated movement velocity by removing signals with frequencies less than a threshold frequency; and
estimating the movement trajectory based on the filtered movement velocity.

18. The system of claim 17, wherein the at least one processor is further configured to use the statistical model to determine the type of activity during the movement of the subject and/or to determine one or more kinematic characteristics during the movement of the subject, based in part on the movement velocity and trajectory.

19. The system of claim 17, wherein the at least one processor is configured to filter the estimated movement velocity by using a movement kinematic machine learning model.

20. The system of claim 1, wherein the one or more kinematic characteristics comprise one or more of: a number of repetitions, a pace of repetitions, an overall consistency, a velocity, a trajectory, and/or a range of motion.

21. The system of claim 1, wherein the statistical model comprises an exercise identification statistical model and a kinematic characteristics statistical model.

22. The system of claim 21, wherein one or more layers of the exercise identification statistical model are connected to one or more layers of the kinematic characteristics statistical model.

23. The system of claim 21, wherein the exercise identification statistical model and the kinematic characteristics statistical model each comprise a respective dilated convolutional neural network.

24. A method for activity identification and characterizing movement kinematics, the method comprising, by at least one processor:
receiving sensor data from two or more sensors, the sensor data indicating movement of two or more body portions of a subject during movement of the subject;
using a statistical model to determine a type of activity during the movement of the subject based in part on the sensor data; and
using the statistical model to determine one or more kinematic characteristics during the movement of the subject, based in part on the sensor data and the type of activity.

25. The method of claim 24, wherein the statistical model simultaneously determines the type of activity and the one or more kinematic characteristics.

26. The method of claim 24, wherein the two or more sensors are attachable to one or more pieces of exercise equipment.

27. The method of claim 24, wherein the two or more sensors are wearable on one or more body portions of the subject.

28. The method of claim 24, wherein:
the sensor data comprises at least first data and second data respectively received from the at least two sensors, wherein the first data and the second data are time-synchronized.

29. The method of claim 28, wherein the at least two sensors include a first sensor attached to a proximal portion of the subject and a second sensor attached to a distal portion of the subject.

30. The method of claim 29, wherein:
the proximal portion of the subject comprises a torso, a thigh, or a proximal portion of a limb of the subject; and
the distal portion of the subject comprises an end of a limb of the subject.

31. The method of claim 29, wherein the sensor data comprises at least a difference between a first reading from the first sensor and a second reading from the second sensor.

32. The method of claim 24, further comprising:
using the statistical model to additionally determine at least a start point and/or a stop point during the movement of the subject.

33. The method of claim 24, wherein the sensor data includes at least one or more of: acceleration, angular velocity, or magnetometer data.

34. The method of claim 24, further comprising, after using the statistical model to determine the type of activity and the one or more kinematic characteristics, using the statistical model to redetermine the type of activity based in part on the one or more kinematic characteristics.

35. The method of claim 24, wherein the type of activity is determined from a predetermined list of activities.

36. The method of claim 32, wherein using the statistical model to additionally determine at least a start point and/or a stop point comprises using the statistical model to determine a start point and/or a stop point of an activity.

37. The method of claim 24, wherein using the statistical model to determine one or more kinematic characteristics comprises estimating movement velocity and trajectory from the sensor data.

38. The method of claim 37, wherein estimating movement velocity and trajectory comprises:
estimating the movement velocity based on the sensor data;
filtering the estimated movement velocity by removing signals with frequencies less than a threshold frequency; and
estimating the movement trajectory based on the filtered movement velocity.

39. The method of claim 38, further comprising using the statistical model to determine the type of activity during the movement of the subject and/or one or more kinematic characteristics during the movement of the subject, based in part on the movement velocity and trajectory.

40. The method of claim 38, further comprising filtering the estimated movement velocity using a kinematic characteristics statistical model.

41. The method of claim 24, wherein the one or more kinematic characteristics comprise one or more of: a number of repetitions, a pace of repetitions, an overall consistency, a velocity, a trajectory, and/or a range of motion.

42. The method of claim 24, wherein the statistical model comprises an exercise identification statistical model and a kinematic characteristics statistical model.

43. The method of claim 42, further comprising passing information between one or more layers of the exercise identification statistical model and one or more layers of the kinematic characteristics statistical model.

44. The method of claim 42, wherein the exercise identification statistical model and the kinematic characteristics statistical model each comprise a respective dilated convolutional neural network.

45. A method for training a machine learning model configured to determine at least a type of activity and one or more kinematic characteristics from a movement of a subject based on sensor data associated with the movement of the subject, the method comprising:
receiving training sensor data collected by two or more sensors, the training sensor data indicating movement of two or more body portions of one or more subjects while performing one or more activities;
obtaining data indicating at least a type of activity and one or more kinematic characteristics performed by the one or more subjects while the training sensor data is collected; and
training the machine learning model to be configured to determine at least a type of activity and one or more kinematic characteristics from a movement of a subject, by using the received training sensor data and the data indicating the at least a type of activity and one or more kinematic characteristics associated with the training sensor data.

46. The method of claim 45, wherein training the machine learning model comprises training an exercise identification statistical model to determine the type of activity from the movement of the subject by using the received training sensor data and the data indicating the type of activity associated with the training sensor data.

47. Them method of claim 46, further comprising:
after training the exercise identification statistical model, locking weights of the exercise identification model; and
training a kinematic characteristics statistical model to determine the one or more kinematic characteristics from the movement of the subject and the data indicating the one or more kinematic characteristics associated with the training sensor data.

48. The method of claim 47, further comprising:
after training the kinematic characteristics statistical model, unlocking the weights of the exercise identification model; and
training the exercise identification statistical model and the kinematic characteristics statistical model using the received training sensor data and the data indicating the at least a type of activity and one or more kinematic characteristics associated with the training sensor data.

49. The method of claim 45, wherein the received training sensor data comprises raw data recorded from an IMU sensor.

50. The method of claim 45, wherein the received training sensor data comprises filtered data recorded from an inertial measurement unit (IMU) sensor.

51. The method of claim 45, wherein the two or more sensors are attachable to one or more pieces of exercise equipment.

52. The method of claim 45, wherein the two or more sensors are wearable on two or more body portions of the one or more subjects.

53. The method of claim 45, wherein:
the training sensor data comprises at least first training data and second training data respectively received from the at least two sensors, wherein the first training data and the second training data are time-synchronized.

54. The method of claim 53, wherein the training sensor data comprises at least a difference between corresponding values in the first training data and the second training data.

55. The method of claim 45, further comprising:
obtaining training start/stop data comprising one or more start points and/or one or more stop points in the training sensor data, wherein the one or more start points each indicates a start of an activity when the training sensor data is collected, and wherein the one or more stop points each indicates a stop of an activity when the training sensor data is collected; and
training the machine learning model to additionally determine at least a start point and/or a stop point during the movement of the subject, by additionally using the training start/stop data.

56. The method of claim 55, wherein obtaining the training start/stop data comprises timing a start and/or stop of an activity performed by a respective subject of the one or more subjects when the training sensor data indicating movement of the respective subject is collected.

57. The method of claim 45, wherein the training sensor data includes at least one or more of: acceleration, angular velocity, or magnetometer data.

58. The method of claim 45, wherein the machine learning model comprises a dilated convolutional neural network.

59. The method of claim 45, further comprising, training the machine learning model to be configured to redetermine the type of activity based in part on the one or more kinematic characteristics.

60. The method of claim 45, wherein training the machine learning model comprises training the machine learning model to be configured to determine the type of activity from a predetermined list of activities.

61. The method of claim 45, wherein training the machine learning model to be configured to determine at least the type of activity and one or more kinematic characteristics comprises training the machine learning model to estimate movement velocity and trajectory.

62. The method of claim 61, wherein training the machine learning model to estimate movement velocity and trajectory comprises:
training the machine learning model to estimate the movement velocity based on the training sensor data and data indicating one or more kinematic characteristics;
filtering the estimated movement velocity by removing signals with frequencies less than a threshold frequency; and
training the machine learning model to estimate the movement trajectory based on the filtered movement velocity and the data indicating one or more kinematic characteristics.

63. The method of claim 62, further comprising training the machine learning model to determine the type of activity during the movement of the subject and/or one or more kinematic characteristics during the movement of the subject, based in part on the movement velocity and trajectory.

64. The method of claim 45, wherein the one or more kinematic characteristics comprise one or more of: a number of repetitions, a pace of repetitions, an overall consistency, a velocity, a trajectory, and/or a range of motion.

65. The method of claim 45, wherein the machine learning model comprises an exercise identification statistical model and a kinematic characteristics statistical model.

66. The method of claim 65, wherein one or more layers of the exercise identification statistical model are connected to one or more layers of the kinematic characteristics statistical model.

67. The method of claim 65, wherein the exercise identification statistical model and the kinematic characteristics statistical model each comprise a respective dilated convolutional neural network.

68. Non-transitory computer readable memory including processor executable instructions that when executed by one or more processors perform the method of any one of claims 24-67.
